(19) United States
(12) Patent Application Publication
(10) Pub. No.: US 2025/0187055 A1
(43) Pub. Date: Jun. 12, 2025

(54) THE INDUSTRIAL DIGESTIVE SYSTEM:" HARDWARE SUPPLY CHAIN ABLE TO PRODUCE BIOGAS, BIOFUEL, CHEMICALS AND REUSABLE CONGLOMERATE/COMPOSITE OUTPUT MATERIAL FROM POTENTIALLY CONSIDERABLY UNCATEGORIZED & MISCELLANEOUS BIOMASS, WASTE, MOLECULES AND ELEMENTS, INTEGRATED WITH SOFTWARE MACHINE LEARNING SYSTEM AND OPTIMIZATIONS ALGORITHMS FOR ENHANCED CONVERSION OF MOLECULAR, ELEMENTAL, AND WASTE MATERIALS INTO VALUABLE RESOURCES."

(71) Applicant: Amedeo Scaramella, Napoli (IT)

(72) Inventor: Amedeo Scaramella, Napoli (IT)

(73) Assignee: Amedeo Scaramella, Napoli (IT)

(21) Appl. No.: 18/972,929

(22) Filed: Dec. 7, 2024

Related U.S. Application Data
(60) Provisional application No. 63/607,842, filed on Dec. 8, 2023.

(51) Int. Cl. B09B 3/60; B09B 3/40; C08K 5/00; C10B 53/00; C12M 1/00; C12N 9/20; C12N 9/26

(52) U.S. Cl. CPC B09B 3/60; B09B 3/40; C08K 5/0091; C10B 53/00; C12M 47/10; C12N 9/20; C12N 9/2411

(57) ABSTRACT

The “Industrial Digestive System” encapsulates a novel paradigm in waste management and resource recovery, bridging the gap between sophisticated computational intelligence and industrial automation. The hardware component replicates the natural digestive system's efficiency in processing a diverse range of inputs, including municipal and industrial waste, transforming them into valuable outputs like biofuels, chemicals, and advanced composite/conglomerate materials. This process is achieved through a series of mechanized operations. Complementing the hardware, the software facet of the invention is rooted in advanced machine learning and optimization algorithms that optimize multivariable functions for the most efficient conversion of input to resources, orchestrating the transformation from varied waste streams, elements, and molecules into solid, liquid, and gaseous outputs. The result is an automated, intelligent manufacturing machine that enhances product quality and operational efficacy. “The Industrial Digestive System” can be seen as a new branch of cybernetics, a cyber-physical system inspired by nature.

### HARDWARE: FIELD OF THE INVENTION

The invention disclosed herein relates to biomass & miscellaneous waste treatment, specifically, organic, paper and uncategorized & miscellaneous waste and generally heterogeneous or homogeneous materials. Inter alia process for transforming considerably percentage of biomass with other uncategorized industrial and/or municipal waste in different output: biogas, biofuel, chemicals substances, polyesters and a conglomerate material; commercially available in forms of products.

### BACKGROUND OF THE INVENTION

The macroeconomic trends for the coming years will essentially concern the ecological sustainability of processes, the recycling of materials, the use of renewable and environmentally sustainable energy sources, as well as an eco-friendly approach to major industrial processes. The circular economy is undoubtedly an essential reality to ensure sustainable and economically profitable material and production processes for the present and to ensure a different sensitivity to large industrial productions for the future. The idea of reusing waste materials and generic waste, especially biomass but also generic industrial and/or municipal waste products, is derived from quantitative and qualitative analysis. The U.S. Environmental protection agency reported that in 2012 Americans generated about 251 million tons of trash and recycled and composted 87 million tons of this material, equivalent to a 34.5 percent recycling rate. About 29 million tons of trash, the 12%, is combusted for energy recovery. The remaining part of waste material, quantified in 135 million tons, is destined to deposit for years in landfills, very often in nature, representing a significant problem for the ecosystems of the entire globe, including the marine ones. The production of greenhouse gases such as methane mainly makes landfill disposal an even more urgent issue. It is of fundamental importance, therefore, the elaboration of an industrial process that recovers the waste materials obtaining energy, in the form of biogas and biofuel, with a combination of processes like depolymerization, hydrolysis and fermentation. These latter are capable of transforming biomass and other materials and bringing it back into the market. At the same time, the process must be sustainable and economically profitable. The idea of creating a digestive system similar to humans or animals is derived from the concept that in nature, everything is reused countless times, creating a perfect circle of life and support for other species and the ecosystem. Numerous processes and techniques aimed at obtaining solutions to this problem and inventors have followed one another during the present millennium, and solutions to the problem of landfills and the recycling of waste material have been developed in recent years. It is essential to mention, for example, solutions with particular attention to the use of hydrolysis and fermentation processes suitable for waste materials and rubbish, solutions for the extraction of biogas and biofuels from waste material, solutions regarding methods and equipment for the processing of waste materials, solutions concerning the processing of resins and thermoplastic materials and the production of reusable products.

U.S. Pat. No. 8,877,992 describe a method for conversion of waste and other organic feedstock into sustainable energy, feed, fertilizer, and other useful products using water, heat, and pressure. The invention provides methods and apparatus that handle mixed streams of various feedstocks like agricultural waste, biological waste, municipal solid waste, municipal sewage sludge, shredder residue to yield gas, specialty chemicals, carbon solids, etc. Despite the large number of organic substances to be handled, U.S. Pat. No. 8,877,992 does not provide a combination of useful processes in order to obtain high-performance products such as fermentation or mixing of solid materials with mechanical forces. Moreover, this invention is difficult to scale due to the large number of separation steps of solid and organic liquid substances in the process. U.S. Pat. No. 9,914,256, who is related with U.S. Pat. No. 8,202,918 describe a method and system for processing waste materials and for manufacturing composite materials. The heterogeneous waste, including various types of waste such as plastic etc., are heated to melt at least a portion of the said plastic component and reduce a volume of said heterogeneous waste, and then mixed until at least some pieces are encapsulated by the melted plastic component, notwithstanding the large number of heterogeneous substances to be handled such organic and paper materials, and in general MSW, U.S. Pat. No. 9,914,256 does not provide a combination of useful processes in order to obtain high-performance products such as fermentation, pyrolysis, hydrolysis and separation between solid and liquid, which is notable in order to obtain optimum high-value energy products like biofuel, biogas and chemical substances. U.S. Pat. No 2019/0375664 provide a method of processing MSW comprising the step of providing a stream of unsorted MSW to a microbial fermentation reactor in which the MSW is fermented with agitation with conditions sufficient to maintain a live lactic acid bacteria concentration of at least 10,000,000,000 CFU/L. Despite the large number of inorganic substances to be handled, U.S. Pat. No 2019/0375664 does not provide a combination of useful processes in order to obtain high-performance products such as pyrolysis or mixing of solid materials with mechanical forces at a temperature above 80° C. moreover this invention is difficult to scale due to the large number of live lactic acid bacteria concentration which must survive with precise environmental conditions for several hours which conditions can be difficult to apply in some contexts. W.O. 2013/187787 provide a method of conducting a pyrolysis process of plastic waste and/or rubber waste and/or organic waste and the use of a chemical modifier in this method comprising subject these components to thermal decomposition in the pyrolytic reactor at a temperature of 200 to 850° C. Notwithstanding the large number of heterogeneous substances to be handled such organic materials W.O. 2013/187787, does not provide a combination of useful processes in order to obtain high-performance products such as fermentation and hydrolysis or mixing of solid materials with mechanical forces at a temperature above 80° C. in order to obtain optimum high-value energy products like biofuel, biogas, chemical substances and useful conglomerate materials. W.O. 2016/181392 provide a method and system for processing waste materials to form biogas and/or bioethanol. The method of W.O. 2016/181392 comprises subjecting waste material to different steps of separation in the process. Notwithstanding the large number of heterogeneous substances to be handled, such as organic, inorganic, plastic etc. materials, W.O. 2016/181392 does not provide a combination of useful processes in order to obtain high-performance products such as hydrolysis and pyrolysis or mixing the solid materials with mechanical forces at a temperature above 80° C. in order to obtain optimum high-value energy products like biofuel, biogas, chemical substances and useful conglomerate materials.

### Software: Field of the Invention

The present invention is grounded in the principle that, in mathematics, for a given multivariable function, there generally exists a maximum or a minimum. It relates to the interdisciplinary fields—primarily, but not exclusively—of waste management, waste treatment, industrial process optimization, renewable resource production, and computational intelligence. More precisely, the invention occupies a critical intersection of advanced software integration and machine learning methodologies, with the aim of transforming and adding value to—primarily, but not exclusively—heterogeneous and/or homogeneous waste streams into—again primarily, but not exclusively—composite materials, biofuel, and biogas. Hence, this invention embodies the software embodiment of the mathematical problem seeking to identify the extremum—a minimum or maximum—that solves the multivariable function representing all transformations from an initial set of inputs, be it atoms, elements, molecules, materials, or wastes, to a specified output state. A “desired output” refers to a preferred state of matter, whether solid, liquid, gaseous, or a combination thereof, and is measurable via state-of-the-art instrumentation. As an illustrative, non-limiting example, this could include the creation, through the invention disclosed herein, of marketable outputs such as composite materials, biofuel, and biogas with specific mechanical, chemical, and physical properties derived from an initial state of a single or multiple elements, atoms, molecules, materials, or wastes. Waste Transformation and Resource Recovery: The invention notably advances the field of waste transformation by systematizing the conversion of all types of waste materials, not limited to miscellaneous landfill waste, hazardous substances, and radioactive materials. The system adopts an all-encompassing approach to waste treatment, aiming for maximum resource recovery and minimal remaining waste. Renewable Energy and Advanced Materials: In parallel, the invention fulfills essential roles in renewable energy and advanced material production. By tapping into the intrinsic value within waste or molecules, it enables the creation of sophisticated composite materials, biofuels, and green hydrogen, thus enhancing the sustainability and resilience of material and energy supply networks. Computational Intelligence and Process Optimization: Incorporated within the invention's structure is a complex layer of computational intelligence that applies machine learning algorithms to rigorously examine, foresee, and refine the various chemical processes implicated in waste conversion. This element of the invention is anchored in artificial intelligence, utilizing data to bolster process efficacy, resource deployment, and product excellence. Software-System Integration and Industrial Automation: Furthermore, the invention extends to software-system integration and industrial automation. It is engineered to integrate smoothly with various hardware systems, including but not limited to, industrial chemical facilities, IoT-enabled waste management systems, and autonomous marine vessels. The system exemplifies the Industry 4.0 ethos, heralding a new era of intelligent and automated waste treatment operations. In conclusion, the invention, recognized as “The Industrial Digestive System” marks a transformative integration of machine learning, waste management, and industrial process optimization. It stands as a tribute to the collaborative power of artificial intelligence and environmental guardianship, with the ultimate aim of pioneering a sustainable and resource-efficient future.

### SUMMARY OF THE INVENTION

The present invention reflects the well-known fact that a natural digestive system is able to process a heterogeneous variety of input materials such as organic, inorganic and others. The aim of this invention is to show that an industrial digestive system following the same process and the same basic method of the natural digestive system is able to produce biogas, biofuel, chemicals substances and an output conglomerate material from potentially heterogeneous uncategorized biomass & waste or potentially homogeneous input materials. The present invention concerns the production, the methods of the industrial chain and the apparatus for processing uncategorized e miscellaneous biomass, waste, and low-value materials for creating fuel-oil, fuel-gas, chemicals and a conglomerate output material. Methods and apparatus for handling/processing waste materials are provided by the invention. In some embodiments, the process concern shredding the input material for creating a paste/. Or bolus in the reflection of the digestive system of the invention; grinding the paste for creating a paste made of particulate; optionally drying the particulate paste; potentially act depolymerization, potentially act a hydrolyzed of the paste; potentially act a fermenting of the paste; separating solid, liquid and gas, then separating liquid, oil and volatile for obtaining biofuel and biogas; separating solids; potentially act a polymerization of solids, then potentially act heating and mixing step under transverse forces the solid part of the paste for obtaining a conglomerate material which is optionally grinder to obtained a bio pellet or a powder or a particulate, the conglomerate material has different properties depending on the initial input material; extruder and compressing the conglomerate material for obtained useful products. In further embodiments, the methods may comprise depolymerizing the paste. In further embodiments, the methods may comprise a pyrolysis reactor. In some embodiments, methods may comprise hydrolyzed the paste by enzymatic reaction; In some embodiments, methods may comprise hydrolyzed the paste by Acid reaction in a specific environment; In some embodiments, methods may comprise hydrolyzed the paste with high temperature and pressure. In some embodiments, depending on the percentage of materials, methods may not comprise the hydrolysis step. In some embodiments, methods may comprise fermenting the paste by bacterial fermentation. In some embodiments, methods may comprise fermenting the paste by mixed acid fermentation; In some embodiments, methods may comprise fermenting the paste by artificial fermentation. In further embodiments, the methods may comprise polymerizing the paste. In further embodiments, the methods may comprise a heating and mixing chamber. In some embodiments, the input materials include biomass from agricultural waste. In other embodiments, the input materials include municipal solid waste. In other embodiments, the input materials include industrial waste; In other embodiments, the input materials include non-municipal waste; In other embodiments, the input materials include medical waste; In other embodiments, the input materials include hazardous and toxic waste. In further embodiments, the input material contains a percentage of the materials mentioned above. In other embodiments, the input material contains an uncategorized percentage of the materials mentioned above.

Summary of the Invention The present invention, denominated as “Industrial Digestive System,” is conceived as a groundbreaking software integration, fortified with—primarily, but not exclusively—advanced machine learning algorithms, expressly designed to transform the paradigms of waste treatment and resource recovery. This invention meticulously addresses the myriad of challenges and limitations delineated in the background, ushering in a new epoch of efficiency, sustainability, and intelligent resource utilization. Innovative Waste to Resource Conversion At the core of this invention is a novel algorithmic framework capable of processing a diverse array of—primarily, but not exclusively—waste inputs or—primarily, but not exclusively—molecules or state of matter, ranging from—primarily, but not exclusively—conventional landfill waste to hazardous and radioactive materials. Through the adept application of machine learning and data analytics, the system meticulously analyzes the composition of—primarily, but not exclusively—waste inputs and/or the system not analyzes the composition of waste/molecules inputs to predict the most efficacious—primarily, but not exclusively—chemical, mechanical, physical processes or transformations required to transform initial elements or a mixture of unknow, known or probabilistically known state of matter or—primarily, but not exclusively—waste input or—primarily, but not exclusively—molecules into a desired output and/or state of matter and/or molecule and/or element and/or composite material and/or biofuel and/or biogas. Maximization of Resource Recovery The “Industrial Digestive System” transcends the boundaries of traditional recycling and waste treatment, achieving unparalleled levels of resource recovery. The system is adeptly engineered to optimize the conversion of—primarily, but not exclusively—waste into a spectrum of valuable outputs, including but not limited to advanced composite materials, biofuels, and green hydrogen, thereby mitigating reliance on virgin materials and contributing to circular economy initiatives. Intelligent Process Optimization Embedded within the architecture of the invention is an advanced layer of computational intelligence, which ensures optimization of processing parameters and resource utilization. The system dynamically adapts to variations in—primarily, but not exclusively—waste input compositions, continuously learning and evolving to enhance efficiency and optimize outcomes. This feature is paramount, as it addresses the prevalent issue of process inefficiencies and suboptimal resource recovery in existing—primarily, but not exclusively—waste treatment methodologies. Versatility and Integration with Industrial Hardware The “Industrial Digestive System” is meticulously designed to seamlessly interface with a myriad of industrial hardware configurations, ranging from chemical reactors, IoT-enabled waste management systems to autonomous maritime vessels. This versatility ensures broad applicability and integration across diverse industrial sectors, fostering the adoption of intelligent—primarily, but not exclusively—waste treatment solutions on a global scale. Environmental Stewardship and Sustainability. In alignment with global imperatives for environmental sustainability, the present invention plays a pivotal role in minimizing the environmental footprint of waste treatment processes. By transforming—primarily, but not exclusively—waste into valuable resources and optimizing process efficiency, the system significantly reduces emissions, conserves natural resources, and contributes to a more sustainable future. In summation, the “Industrial Digestive System” stands as a beacon of innovation in the realm of waste management and resource recovery. Through the integration of—primarily, but not exclusively—machine learning, computational intelligence, and advanced software engineering, the invention addresses the critical challenges of existing—primarily, but not exclusively—waste treatment methods, setting a new standard for efficiency, sustainability, and intelligent resource utilization.

### BRIEF DESCRIPTION OF THE DRAWINGS

More particular descriptions of the invention are made by reference to certain exemplary embodiments thereof which are illustrated in the appended figures. These figures form a part of the specification. It is to be noted, however, that the appended figures illustrate exemplary embodiments of the invention and therefore are not to be considering limiting in their scope.

FIG. 1 is a flowchart illustrating an exemplary process according to the present invention.

FIG. 2 is a schematic diagram depicting some exemplary apparat used to perform an exemplary process of the present invention.

FIG. 3 is a flowchart illustrating an exemplary process according to the present invention.

FIG. 4 is a flowchart illustrating an exemplary process according to the present invention.

FIG. 5 is a flowchart illustrating an exemplary process according to the present invention.

FIG. 6 is a flowchart illustrating an exemplary process according to the present invention.

FIG. 7 is a flowchart illustrating an exemplary process according to the present invention.

FIG. 8 is a schematic diagram depicting some exemplary method used to perform an exemplary training process of the present invention.

FIG. 9 is a flowchart illustrating an exemplary algorithm process according to the present invention.

FIG. 10 is a flowchart illustrating an exemplary algorithm process according to the present invention.

FIG. 11 is a flowchart illustrating an exemplary algorithm integration with hardware according to the present invention.

FIG. 12 is a flowchart illustrating an exemplary process according to the present invention.

FIG. 13 is a flowchart illustrating an exemplary process according to the present invention.

FIG. 14 is a flowchart illustrating an exemplary process regarding the outputs.

FIG. 15 is a schematic diagram depicting some exemplary method used to perform an exemplary process of the present invention.

### DETAILED DESCRIPTION OF THE INVENTION IN HARDWARE

Embodiments of the present solution represent a sustainable solution concerning the disposal of a huge amount of low cost and almost always unusable biomass and waste materials. Environmental sustainability regards the utilization of chemical, physical and mechanical processes that are compatible with the low emission of greenhouse gas and low amount of energy necessary. Furthermore, the environmental sustainability provided by this invention reflect the natural processing of materials in a living organism and emulate the complex system inside the digestive apparatus of an animal; this enables the invention to be consistent with the basic idea to process waste input for obtained reusable products, those with an energy value superior respect to the input materials. Moreover, embodiments of the present invention quantitative represent a huge solution concerning the huge amount of greenhouse gas expelled by organic biomass in the landfill, plastics, electronics, metal and other industrial and/or municipal waste that are no longer recyclable or reusable in a specific matter. The profitable sustainability provided by this invention concerns the fact that low-cost products, optionally separated, and waste, optionally separated, are processed in a useful product in the authentic spirit of the circular economy. Furthermore, the present invention provides a waste management solution superiorly, both in the environmental sustainability and profitable sustainability, to other solutions concerning mechanical, biochemical physical or chemical procedure; conventional processes like combustion, incineration, gasification, mechanical heating & mixing and directly pyrolysis, are quantitative inferiorly sustainable respect to the present invention. Samples Products regarding the present invention include syngas, fuel oil, fuel gas, hydrocarbon liquid, different type of bioplastic, chemical substances and a conglomerate solid material; which products can be optionally removed in precise process steps, reused in precise process steps, mixed in precise process steps, such as but not limited to, the use of biogas and/or biofuel for an energy source for the whole process, the mixing of bioplastic products with conglomerate products, the mixing of biogas and biofuels to obtain precise mixtures and other chemicals products. A further advantage of this invention is the absolute and complete use of heterogeneous waste materials and biomass, optionally separated, then in relation to the—know, hypothetic or unknown—percentage of different types of input materials can be processed with different types of mechanical, chemical, biochemical and physical procedures described by this invention, in order to obtain the best products which are based on methodological scientific tests and calculation. For example, in some embodiments of the present invention, knowing the percentage of input materials, a pyrolysis step in the process is optionally used. In other embodiments of the present invention, knowing the percentage of input materials, the hydrolysis step is proved not to be necessary for the process. In other embodiments of the present invention, knowing the percentage of input materials, the fermentation step is proved not to be necessary for the process. In still other embodiments of the present invention, unknowing the percentage of input materials, the hydrolysis, pyrolysis and fermentation steps are proved to be necessary for the process. In still other embodiments of the present invention, unknowing the percentage of input materials, the hydrolysis and fermentation step are proved to be necessary for the process. In still other embodiments of the present invention, unknowing the percentage of input materials, the depolymerization, the hydrolysis, pyrolysis and fermentation step is proved to be necessary for the process. In further other embodiments of the present invention, unknowing the percentage of input materials, the depolymerization step is proved to be necessary for the process. In further other embodiments of the present invention, knowing the percentage of input materials, the depolymerization step is proved to be necessary for the process. The processes described presently are able to manage different types of input materials, knowing or unknowing the percentage and optionally separating those materials, before the other step, in order to obtain useful products. In some embodiments of the processes described presently, managing different types of input materials in percentage mostly composed of organic mass, carbon, lignocellulosic materials required a biochemical conversion process strictly related to a biorefinery or distiller additional step, like a distiller for the ethanol processing. In some embodiments of the processes described presently, managing different types of input materials in percentage mostly composed of plastic, organic mass and MSW materials required a physical conversion process, a biochemical conversion process, that are strictly related to an additional pyrolysis step. In some embodiments of the processes described presently, managing different types of input materials in percentage mostly composed of plastic, metal, industrial, hazardous, and toxic materials required a pyrolysis step process. The myriad of heterogeneous materials that the process described in the present article can process; combined with precise chemical, biochemical and physical processes such as hydrolysis, fermentation and pyrolysis, which in turn are related to a mechanical process of heating & mixing of solid products and refining of liquid and gaseous products, make this an invention far superior to the traditional waste management techniques and technologies used. Moreover, it surprises the ability of some embodiments of this invention to perform adequately in the presence of inputs composed of heterogeneous materials of unknown percentage and in the presence of inputs composed of heterogeneous materials of known percentage and in the presence of inputs composed of homogeneous materials of known percentage. In addition, it is noteworthy the characteristic of this invention concerning adapting the best process and/or the best processes such as hydrolysis, pyrolysis, fermentation, depolymerization, polymerization, distillation, heating, mixing etc., to the percentage—although it is known, unknown or theoretically hypothetical—of heterogeneous or homogeneous materials that compound the input. Also notable and sensational is the ability of some embodiments of the present invention to perform thanks to the energy provided by biofuel or biogas, which are produced by the process itself. The ability to produce, from some embodiments of this invention, an output conglomerate material with biochemical, chemical, physical and mechanical characteristics absolutely reassuring in the application of the same in different contexts; some not limiting examples concern the use of the output conglomerate material for the production of objects of common and industrial use. Such a conglomerate material turns out to be the solid output part of the input materials. Depending on the input materials, it can have, depending on the context, different mechanical, biological, physical and chemical properties, which allow a different application. In some non-restrictive examples, the conglomerate material had thermoplastic characteristics and excellent mechanical performance, which showed a different range of optimal applications like modelled objects of daily use or industrial stocks. In other non-restrictive examples, heating and mixing under transverse forces the solid part of the heterogeneous input to obtain a conglomerate material which is processed to obtain pellet or a powder which are processed in an extruder and compressing for obtaining a wide range of useful products.

### Definitions Hardware & Software

“Digestive system”, as used herein, is related to the animal digestive system, and it is useful to keep it in mind as embodying the inventive power of the present invention; exemplary the fundamental step of the human digestive system who are: Ingestion, Mechanical processing (crushing and shearing), Digestion (the chemical breakdown of food into small organic for absorption by digestive epithelium), Secretion (release of water, acids, enzymes, buffers, salts), absorption (into the interstitial fluid of digestive tract), excretion (removal of waste product from body fluids and defecation process for removing the feces). “Input”, as used herein, refers to any type/s of molecules, elements or substance/s, in liquid, solid or gas state, inserted at the start of the process of the present invention, can be heterogeneous or homogeneous. “Elements,” as used herein, refer to the fundamental chemical substances consisting of atoms that share the same number of protons in their atomic nuclei. Each element is defined by its atomic number-representing the count of protons in its nucleus—and possesses distinct chemical and physical properties. Elements may be encountered in various states of matter (solid, liquid, or gas) and can be present individually or combined with other elements to form more complex substances (e.g., compounds, mixtures, or alloys). In the context of the present invention, “elements” encompass not only stable, naturally occurring elements, but also isotopes, synthetically produced elements, or elemental constituents derived from other materials as they progress through the “industrial digestive system. “Molecule,” as used herein, refers to a group of two or more atoms bonded together, forming the smallest identifiable unit of a compound that retains its chemical properties. In the context of the present invention, molecules may constitute part of the input or result from the transformation processes within the industrial digestive system, serving as fundamental building blocks that can be broken down, reorganized, or combined to create usable outputs. “Output”, as used herein, refers to any type/s of substance/s in liquid, solid or gas state, expelled at some step of the process of the present invention. “Sustainability”, as used herein, is referred to environmental sustainability, economic sustainability, and social sustainability. Environmental sustainability reflects the responsibility to preserve natural resources and protect global and local ecosystems from supporting the health of the biosphere; environmental sustainability englobes energy sustainability, which is, as used herein, the utilization of sources of energy different to the fossil fuel one, exemplary, but are not limited to, the energy produced from waste management, energy from biofuel and biogas. Economic sustainability or profit sustainability, as used herein, concerns the fact that the process described by this invention is quantitatively profitable and therefore scalable as a form of a self-sustaining business. Social sustainability is concerned that the present invention is essentially based on the effective improvement of deep-seated social processes such as the production of various types of organic waste, inorganic and mixed by the human society and waste from different industries dedicated to producing for the latter. Furthermore, this invention reserves the right to be based on the absolute automation of the process, also thanks to artificial intelligence software, to allow speeded up, safe and advantageous processes for the single and the entire society. “Particulate paste” or “bolus” as used herein concerns heterogeneous or homogeneous input substance after being reduced to particulate matter of components less than 0.5 cm by adequate shredding, crushing and grinding mechanical machine. “biomass”, as used herein, refers to any type of organic matter, organics materials derived, directly or indirectly from animals and plants. “Conglomerate/composite material”, as used herein, refers to heterogeneous/homogeneous solid output material with different biological, chemical, physical, and mechanical properties, which in turn depend on the composition percentages of the materials at the input stage. A non-limiting example regards the thermoplastic elements and mechanical properties that the conglomerate material possesses. A non-limiting example regards the properties of the organic elements concerning textile fibers that the conglomerate material possesses. A non-limiting example regards the mechanical properties of carbon components in the conglomerate material. “Heterogeneous waste” as used herein concerns the ability of the process of this invention to be able to perform with different types of waste materials for example but not limited to: industrial waste, agricultural waste, municipal waste, commercial waste, Sewage sludge, Radioactive waste, Medical waste, paper, glass, metal, plastic (PETE, HDPE, polypropylene, PET, P.P., LDPE, PVC, P.S., polystyrene, film products, etc.), electronics, organics (food, leaves, grass, pruning, trimmings, branches, stumps, manures, lumber, wood, pallets, crates, agricultural waste, forest waste, farming waste etc.), inert material (piece of building foundation, paving, blocks, asphalt paving, asphalt roofing, gypsum board, rocks, drywall, carpet, soil, fines, stones, sand, brick, ceramics, fiberglass, mixed demolition debris, paint, auto batteries, lubricating oil, batteries, gas cylinder), Hazardous waste (pills, liquid creams and pharmaceuticals, household hazardous, pesticides, LED lamps, mercury containing items, vehicles and equipment fluids, caustic cleaners, fluorescent lamps), special waste (tires, bulky items, mattresses and foundations, ash, auto bodies, medical waste), miscellaneous (organic textiles, synthetic textile, shoes, purses, belts, solar panel, diapers, sanitary products, garden hoses, cigarette butts, cosmetics, straw basket, animal carcasses, mixed residue, shredding residue, feces, rubber sports balls, MRF residual fines, kitchen ceramics, synthetic rubber products). “Homogeneous waste”, as used herein, refers to materials of the same kind of input consisting of parts all of the same kind. “lignocellulosic”, as used herein, refers to lignocellulosic biomass, which is composed of two kinds of carbohydrate polymers, cellulose and hemicellulose, and an aromatic-rich polymer called lignin. “Municipal solid waste (MSW)”, as used herein, regarding waste type consisting of everyday items that are discarded by the public. The composition of such MSW varies greatly from municipality to municipality and change significantly with time. A typical non-limiting example of MSW concern: biodegradable waste (food and kitchen waste, green waste, paper), recyclable materials (paper, cardboard, glass, bottles, jars, tin cans, aluminum cans, aluminum foil, metals, certain plastic, textiles clothing, tires, batteries etc.), Inert waste (rocks, debris, dirt, construction and demolition waste), electrical and electronic waste (electrical appliances, light bulbs, washing machines, T.V.s, computers, screens, mobile phones, alarm clocks watches, etc.), composite wastes (waste clothing, tetra pack food and drink cartons, waste plastics, toys, etc.), Hazardous Waste (paints, chemicals, tires, batteries, light bulbs, electrical appliances, fluorescent lamps, aerosol spray cans, fertilizers, etc.), Toxic waste (pesticides, herbicides, fungicides), biomedical waste(pharmaceutical, drugs, etc.). A non-limiting example in table 1 shows the difference, in percentage, between the MSW composition in China and in the U.S.

TABLE 1

| | Food/ | | | | Wood | | |
|---|---|---|---|---|---|---|---|
| MSW | organic | paper | plastics | textiles | waste | rubber | Other/miscellaneous |
| The | 33% | 20% | 14% | 3.6% | — | — | 29.4% |
| U.S. | | | | | | | |
| CHINA | 55% | 8.5% | 11.2% | 3.2% | 2.9% | 0.8% | 18.4% |

“Industrial waste”, as used herein, concerns the two types of industrial waste: hazardous and non-hazardous waste. Non-hazardous industrial waste is the waste from industrial activity, which does not represent a threat to the public or the environment. Hazardous waste is a residue from industrial activity that can damage public health and the environment. Non-limiting examples are shown in table 1. Source: Ria Millati PhD, Mohammad J. Taherzadeh PhD, in Sustainable Resource Recovery and Zero Waste Approaches, 2019.

TABLE 2

| Sector | Subsector | Examples | Waste Products |
|---|---|---|---|
| Mining- | | Extraction; beneficiation, | Solid rock, slag, |
| Quarrying | | processing of minerals | phosphogypsum, muds, |
| | | | tailings |
| Energy | | Electricity, gas, steam, | Fly ash, bottom ash, boiler |
| | | and air-conditioning | slag, particulates, used oils, |
| | | supply | sludge |
| Manufacturing | Chemical | | Spent catalyst, chemical |
| | | | solvents, reactive waste, acid, |
| | | | alkali, used oils, particulate |
| | | | waste, ash, sludge |
| | Food | | Plastic, packaging, carton |
| | Textile | | Textile waste, pigments, |
| | | | peroxide, organic stabilizer, |
| | | | alkali, chemical solvents, |
| | | | sludge, heavy metals |
| | Paper | | Wood waste, alkali, chemical |
| | | | solvents, sludge |
| Construction | | Construction; demolition | Concrete, cinder blocks, |
| | | activity | gypsum, masonry, asphalt, |
| | | | wood shingles, slate, metals, |
| | | | glass, plaster |
| Waste/Water | | Water-collection; | (No waste products listed) |
| Services | | treatment, and supply | |

“Agricultural/farming waste”, as used herein, is unwanted or unsalable materials produced from agricultural/farming operations. “Radioactive waste”, as used herein, is a type of hazardous waste that contains radioactive material. “Medical waste”, as used herein, is a subset of wastes generated at health care facilities, such as hospitals, physician offices, dental practices, veterinary hospitals, clinics, laboratories etc. “low-value materials” as used herein are unwanted or unsalable materials or generic material with a low-value price determined by the market. “bioplastic”, as used herein, is a type of biodegradable or non-biodegradable plastic derived from biological substances. “percentage %” as used herein, percentage relates to a part of an input established in 100 units of mass. The percentage of materials that can constitute the input can be known, unknown or hypothetical and can refer to both homogeneous inputs and heterogeneous inputs. According to the percentage and its knowability, it is the task of an ordinary expert in the art to understand the best or the best combinations of processes feasible in order to obtain from an input the desired output product. “Applied forces” as used herein, all the forces artificially applicable or naturally applied and exploitable to give dynamism to the process in its totality, they can for—non-limiting—example include conveyors, gravity, hydraulic properties, pressure, mechanical forces, elastic forces, etc. “environment” as used herein, set of physical, chemical, biological, mechanical or combination characteristics and variables of the above, to the whole of a given chamber or more chambers or reactors of the second stage. Such variables as, for example, but not limited to, rooms with a certain temperature, pressure, P.H., percentage of water, solvents or additives, presence of biological forms such as bacteria and fungi of various nature and kingdom, presence of different types of enzymes, presence of volatile components such as oxygen, nitrogen, carbon dioxide, methane, ethane and time. “CM conglomerate material”, as used herein, refers to the final solid product of the process of the present invention. “Fuel oil”, as used herein, refers to one of the final liquid products of the process of the present invention. “Fuel gas”, as used herein, refers to one of the final volatile products of the process of the present invention. “Chemical substances”, as used herein, refers to final volatile and liquid products of the process of the present invention that are not fuel gas or fuel oil.

### Overview of the Process in Hardware

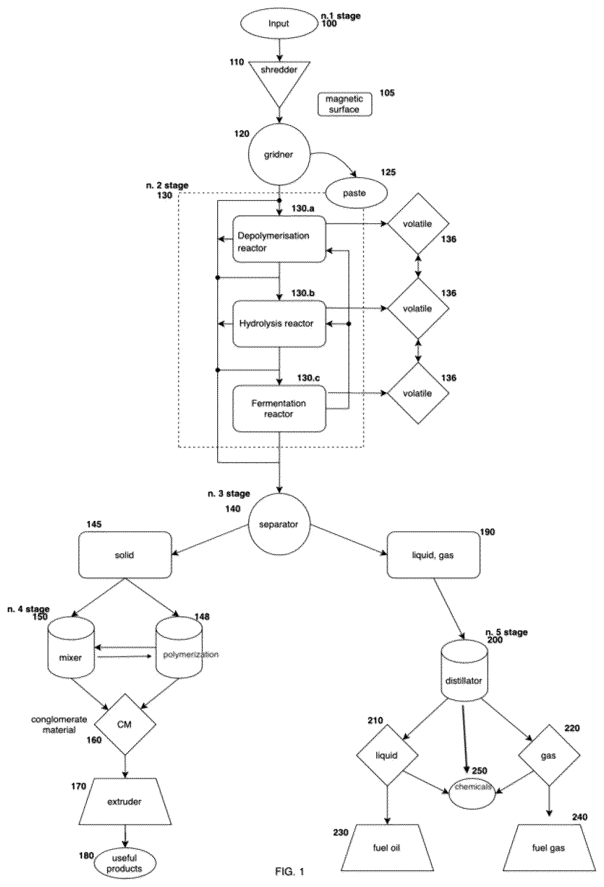

Embodiments of the present invention convert heterogeneous waste (the input) into fuel, gas, conglomerate material, and other useful products (the output) using mechanical processes such as shredding, crushing and grinding, chemical process as hydrolysis, biochemical process as fermentation and physical process as depolymerization or pyrolysis, using heat, mixing and different applied forces, non-limiting example include pressure, hydraulic forces, transversal forces of gravity, in different steps. In certain embodiments, the system can also accept non-waste inputs, including molecular or elemental feedstocks or other raw materials with defined compositions and properties. In such cases, the processing steps and conditions-optimized through machine learning and adaptive control—are similarly applied to these well-characterized feedstocks to produce valuable outputs efficiently. Embodiments of the present invention have the ability, thanks to a system of pumps, pressure and mobile doors, to process the heterogeneous waste input in different steps, which may be optionally used or omitted depending on whether the input percentage of different materials material is known, unknown or hypothetical, in order to obtain the optimum output products in relation to the input compositions. Typically, the heterogeneous waste input—with a percentage of different materials that is known, unknown or hypothetic—is processing into a paste with the utilization of mechanical forces like shredding, crushing and grinding machine, after is optionally pumped, thanks to applied forces, into a pyrolysis reactor which is able to heat and pressure the paste, secondly is optionally pumped into an hydrolysis reactor, which is able to chemical breakdown the major components of the input materials as macromolecules in smaller simpler molecules or monomers, than is optionally pumped into a fermentation reactor which is able to process the paste and, depending of the percentage of the input, obtain useful output like ethanol, methane, lactic acid, bioplastic etc., after these steps, that can be optionally used or omitted depending of the knowability of the heterogeneous input percentage, the processed paste is separated in solid, liquid and gas components thanks to separator as filters or centrifuges, the liquid and gas components are pumped into a refinery or distillery or in equipment useful to the process of extraction of gas or fuel, absolutely understandable to a skilled in the art, the solid part is optionally pumped in a vessel for the polymerization or pumped into an heating & mixing machine which process the conglomerate material, whom has different mechanical, chemical and physical properties depending on the percentage of the input materials, the conglomerate material is than processed into a pallet or powder and thanks to an extruder modelled in useful products. In some embodiments of the present invention, the vessels for pyrolysis, hydrolysis and fermentation are multiple and in parallel and/or in series and are able to incorporate different biological, chemical, mechanical and physical reactions in order to obtain the desired product in relation to the different percentage of input materials. A non-limiting example regarding multiple vessels for hydrolysis which contains a mix of acids, a cocktail of enzymes, thermal and pressure characteristics in order to adopt the best type of process to the considered input material. A non-limiting example regarding multiple vessels for fermentation which contains more than one type of cocktail of bacteria in a different environment with different characteristics like temperature and PH and different vessels which possess different properties, characteristics, and processes such as condensation, pressure, time, and thermal characteristic, in order to adopt the best type of process in relation to the considered input material. An ordinary skilled in the art can easily deduce the best hydrolysis process, fermentation process and pyrolysis process or the best combination of hydrolysis, fermentation and pyrolysis based on the percentage—whether it is known, unknown or hypothetical—of input materials, in order to obtain, considering the chemical, physical and biological variables of the case, the desired output products. An ordinary skilled in the art can easily deduce the best hydrolysis process such as acid hydrolysis, enzymatic hydrolysis or thermal hydrolysis based on the percentage—whether it is known, unknown or hypothetical—of input materials, in order to obtain, considering the chemical, physical and biological variables of the case, the desired output products. An ordinary skilled in the art can easily deduce the best fermentation process such as the optimum bacterial cocktail or the most efficient artificial fermentation based on the percentage—whether it is known, unknown or hypothetical—of input materials, in order to obtain, considering the chemical, physical and biological variables of the case, the desired output products. An ordinary skilled in the art can easily deduce the best pyrolysis process such as the optimum temperature, time, pressure, etc. and the most efficient combination of the latter, based on the percentage—whether it is known, unknown or hypothetical—of input materials, in order to obtain, considering the chemical, physical and biological variables of the case, the desired output products. High-level block diagrams of exemplary embodiments of the invention are provided in FIG. 1, and more detailed illustration, specific procedure and apparatus of exemplary embodiments are presented in the following step and described in detail below.

### Paste Preparation

Embodiments of the present invention can process heterogeneous or homogeneous waste materials without a pre-separation of that materials and, based on the percentage—whether it is known, unknown or hypothetical—of input materials can adopt the best procedure in order to obtain the desired product. In some embodiments of the invention, as illustrated by the figures, the Input 100, used in accordance with the definitions of the present invention, is subjected to mechanical forces like shredding the machine 110 and Grinding machine 120 in order to obtain a heterogeneous particulate of materials which form a paste. In some embodiments of the invention, as illustrated by the figures, the Input 100 may also include non-waste raw materials, such as molecular or elemental feedstocks, enabling the system to broaden its applicability beyond traditional waste streams, enhancing versatility and resource efficiency. These non-waste inputs are treated using the same adaptive processes designed to optimize output quality and yield. The scope of the paste preparation step is to reduce the volume of the waste materials. Unlike the volume occupied by waste materials, this paste is enormously more modulable and workable, and it is also easily transferable thanks to pumping systems inside pipes and applied forces. A further purpose of the mechanical process of disintegration of heterogeneous material is the advantage that the paste can be subjected to chemical, physical, mechanical or biological processes in a simpler way and with a great advantage in terms of timing and performance. In order to obtain simplicity and speed of the process, it is possible that a liquid, such as water, is added to this paste, which makes it transportable to the second step 130 with simpler modes and can be attached to a hydraulic complex. In some non-exhaustive examples, the input material, composed of a known percentage of organic material, was adequately Shredder and grinded into a particulate composed of pieces of material less than 0.5 cm, such pre-processing was enormously advantageous before exporting the material to the whole of a hydrolysis chamber, containing a specific cocktail of enzymes such as a large number of Amylase enzymes at a temperature of between 40° C. and 50° C. and with a neutral PH. A possible mixture of liquids and gases released in the mechanical shredding and grinding process is contained inside hermetic containers and reinserted inside the paste. There is, therefore, no leakage of liquids or volatile substances in the first stage. As for the metal, one or more magnetic surfaces are positioned between or after steps 100, 110,120 with the objective of extracting the greatest number of metal substances thanks to the magnetic forces. These metal substances constitute a first output product and can be optionally reinserted as a whole into the processor directly transported to a foundry or appropriate places that will be well known to a skilled in the art. Depending on the different characteristics and percentages of the input materials, the process of creating the paste may therefore require the addition of liquids such as water or specific solvents/additives between or after steps 100, 110,120 with the aim of making the heterogeneous input compound more manageable. The preparation of the paste in the first stage takes place in static or mobile/rotational mechanical chambers equipped with cutting blades of different thicknesses, sizes etc., and of different compositions such as steel or titanium, and different mechanical procedures to reduce the input material in a semi-solid compound that is easily transportable thanks to tubes of considerable thickness and subjected to physical forces applied such as, but not limited to, lateral pressures, gravity, hydraulic principles. This semi-solid compound or paste contains heterogeneous liquids, solid and gaseous materials, which are kept in the industrial chain thanks to hermetic tubes and able to maintain their rigidity even in the presence of significant pressures. This paste can also be obtained with the aid of multiple shredding machines and multiple grinder machines, with the aim of obtaining a semi-solid compound formed by a mixture of heterogeneous base elements of thickness less than 0.5 cm. A skilled ordinary in the art can easily deduce the best size of the basic elements related to the percentage of input materials in order to obtain the best performance from the processes of hydrolysis, fermentation and pyrolysis. The paste, after being properly processed by mechanical forces, can either directly be transported from the pipe system to the entire chamber for the second stage be temporarily stored in order to reach a volume or weight established for the processes in the second stage. The multiple numbers of tubes and movable doors, which can be operated either manually by an operator, or thanks to computerized systems, allows wide freedom in the process and also the concrete achievement of a precise path that such paste must cover in order to obtain the best product in relation to the percentage—that it is known, unknown or hypothetical—of heterogeneous input materials. Some not limited examples are to knowing a percentage of input materials composed of more than 80% organic material, which has undergone the appropriate mechanical processes and has been reduced to a semi-solid mixture or paste, has been transported, thanks to a hydraulic system of pressure hoses and a small number of doors operated by an operator, to the whole of a hydrolysis chamber containing numerous enzymes which have excellently preformed the same system of hydraulic pumps, after a time understandable by an ordinary skilled in the art, transported the hydrolyzed paste including liquid, solid and gaseous substances, in a room suitable for bacterial fermentation; subsequently, the same pressurized hydraulic system was excellently able to transport the fermentation products to the whole of a separation chamber equipped with numerous filters and a centrifuge, which separated the solid materials from the liquid and gaseous ones. The possibility of transporting the paste inside hermetic tubes, able to avoid the dispersion of potentially harmful and toxic substances, thanks to the use of applied forces and to operate movable doors thanks to an operator and/or a computer, allows extreme sustainability, as defined in advance, security and flexibility of the entire process as well as a maximum yield in the production of useful products. It is easily deduced from an ordinary skilled in the art, what is the best percentage of liquids and the best forces applicable in order to easily transport the paste into the pipe system and thus prevent problems related to extreme friction between the parts and limitations in terms of viscosity as well as a use excessive use of energy resources.

### Apparatus

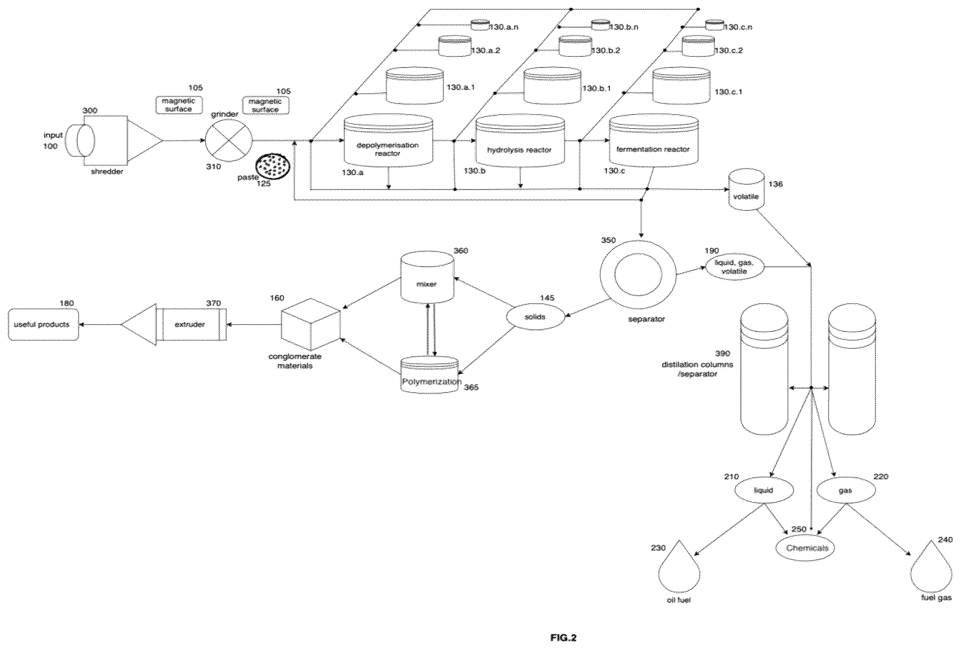

The preparation of the paste by mechanical grinding forces is the first step 300 of the process, which can be appreciated from the diagram in FIG. 2. the next step 310 concerns the use of grinders, which may have different characteristics associated with the manufacturer of such machines, such characteristics may include, for example, energy expenditure in KV/h, the material from which the blades are made, the time necessary to obtain particles of a certain section, the rotational period etc., an ordinary skilled in the art is able to appreciate the best type of machinery that can be used to optimize the process in its entirety. In some embodiments of the present invention, there are specific machines such as for example conveyors with magnetic surfaces that can attract the greatest number of metal substances prior to the second stage. In order to obtain a performant process in the transport of this paste 125 in the following apparatuses provided for in the second stage, it is possible and optional to add liquids to this composition or to exploit in the most optimal way forces applied as gravity, pressure and hydraulic properties, an ordinary skilled in the art is able to understand the necessary density and viscosity of the paste to be easily transported in subsequent apparatuses and the possible aid of applied forces. In the diagram in FIG. 2, it is possible to appreciate some nodes placed on the directed lines joining the different steps; these nodes represent arbitrarily opening doors that allow using the best process or the best combination of processes in order to obtain the desired product in relation to the percentage of input materials; an ordinary skilled in the art is able to understand the best arbitrary path that the said paste must make in order to be used in the most optimal way for the production of usable products.

### Second Stage: Depolymerization

Referring to FIG. 1, the paste 125, obtained from the mechanical processes previously mentioned 110, 120, is transported in the second stage 130; it can be arbitrarily directed into a specific compartment for the decomposition of materials or depolymerization 130.a. This compartment may be, for example, but not limited to a pyrolysis reactor. The following reactor is based on the process of thermochemical decomposition; it is capable by the application of heat and pressure and in the complete absence of any oxidizing agents, such as oxygen, to break down the original chemical bonds by forming simpler molecules. The paste is heated to induce thermal homolysis at temperatures above 300° C. In some embodiments of the present invention, The reactor has heated the paste to a temperature between 400° C. and 600° C. In some embodiments of the present invention, the gaseous products of pyrolysis were extracted from the reactor and arbitrarily reinserted into some steps of the process. In some embodiments of the present invention, the gaseous products of pyrolysis were not extracted from the reactor but compressed by the use of pressure and addressed together with the semi-liquid paste in the next step. In some embodiments of the present invention, the carbon-rich solid residues of the pyrolysis process previously mentioned were transported directly to the fourth stage of the process, being suitably mixed and heated in step 150 with or without the addition of other solid materials and additives/solvents. An ordinary skilled in the art is able to understand if this process is necessary or omitted in order to obtain the best final product knowing or assuming the percentage of input materials, Some non-restrictive examples concern percentages of organic material input ranging from 70% to 90% resulting in a high composition of cellulose and lignin, which have favoured a pyrolysis process performing at temperatures ranging from 200° C. to 400° C. In some non-limiting examples, the pyrolysis rector working at lower-than-average temperatures has excellently performed as a bio-drying apparatus allowing better characteristics to the final product. After the pyrolysis process are optionally extracted only volatile and gaseous substances, other substances such as, but not limited to, proteins, fats, different types of minerals, metals, ceramics, glass, plastics, solids of various kinds, carbonaceous materials, ash, fibres, organic and inorganic residues, etc. may be incorporated in the semi-liquid paste or deposited on the bottom together with the charcoal residues typical of the hydrolysis process. An ordinary skilled in the art can easily deduce the composition resulting from the hydrolysis process in relation to the input materials used. In some embodiments of the present invention, some solid materials with a higher density than the rest of the semi-liquid paste are deposited on the bottom together with carbon residues of the pyrolysis process due to gravity, such natural separation of substances is optionally used by collecting such substances deposited on the bottom with high density and transporting directly to the fourth stage for further steps of the process, The remainder of the semi-liquid paste obtained in the hydrolysis reactor may be arbitrarily transported in a container for processes such as fermentation and/or hydrolysis and/or directed directly to the third stage 140. A skilled ordinary in the art is able to deduce which is the best step, among those possible, after step 130.a of pyrolysis in relation to the different input materials. The pyrolysis chamber is controlled by an operator and/or a computer capable of arbitrarily and dynamically regulating variables such as, but not limited to: time, pressure and temperature depending on the type of input considered. In some embodiments of the present invention, knowing or assuming the percentage of the materials of input, it has been held opportune to operate with temperatures comprised between the 100° C. and the 350° C., so as to avoid the formation of char and coal, and with pressure in a range between 100 psig and 500 psig and in a time between 120 minutes and 300 minutes. In other embodiments of the present invention, knowing or assuming the percentage of input materials, It was necessary to operate with temperatures between 400° C. and 900° C. and with pressure in a range between 400 psig and 900 psig and in a time between 30 minutes and 90 minutes, in order to obtain a semi-liquid paste rich in char and coal. In still other embodiments of the present invention, knowing or assuming the percentage of input materials, it was appropriate to operate at temperatures between 100° C. and 150° C. and at a pressure of about 100 psig and in a time between 300 minutes and 400 minutes, in such a way as to obtain a semi-liquid paste that can be transported directly to an apparatus used for a fermentation 130.c of microbial digestion in an anaerobic environment. In still other embodiments of the present invention, knowing or assuming the percentage of the input materials, it was appropriate to operate with temperatures ranging between 800° C. and 900° C. and a pressure of about 600 psig and in a time between 15 minutes and 30 minutes, to obtain a semi-liquid paste rich in hydrocarbons transportable directly to the third stage 140. In still other embodiments of the present invention, knowing or assuming the percentage of input materials, It was considered appropriate to operate at temperatures between 850° C. and 1000° C. and with a pressure of about 800 psig and in a time between 35 minutes and 50 minutes, in order to obtain a semi-solid paste rich in coal transportable directly to the fourth stage 150. In still other embodiments of the present invention, knowing or assuming the percentage of input materials, it was appropriate to operate at temperatures between 100° C. and 200° C. and at a pressure of about 300 psig and in a time between 45 minutes and 150 minutes, in order to obtain a semi-liquid paste suitable for the hydrolysis process 130.b. The advantage in heating at such temperatures and with such pressures the paste obtained mechanically from the input materials is evident in the drastic exemplification of some secondary processes necessary in order to obtain the best final products. Such processes as, for example, but not limiting ourselves to, the hydrolysis 130.b, fermentation 130.c and mixing combined with heat and transverse forces 150, results in better performance after the breakdown of macromolecules or different components as polymers of various types and origin. Amino acids are, for example, the product of the breakdown of more complex proteins that can be limiting in subsequent steps. A great advantage in terms of transportability of semi-solid paste is the properties of friction and viscosity that the pyrolysis process can improve in order to transport through pipes operating with applied forces. A skilled ordinary in the art is able to deduce which advantageous hydrodynamic characteristics are obtainable after a hydrolysis process. In still other embodiments of the present invention, depolymerization occurred by providing heat and pressure in modes different from classical pyrolysis.

### Second Stage: Decomposition by Hydrolysis

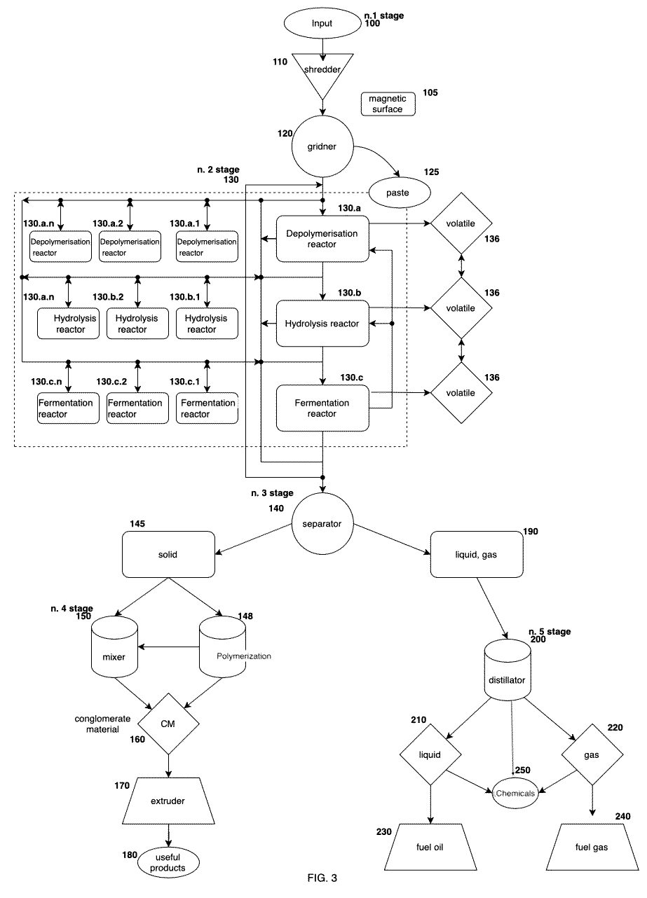

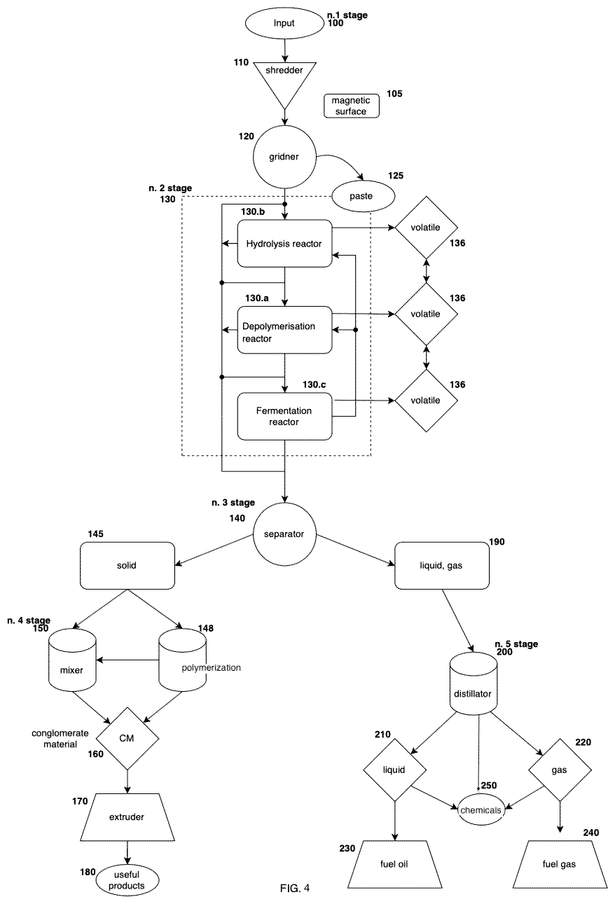

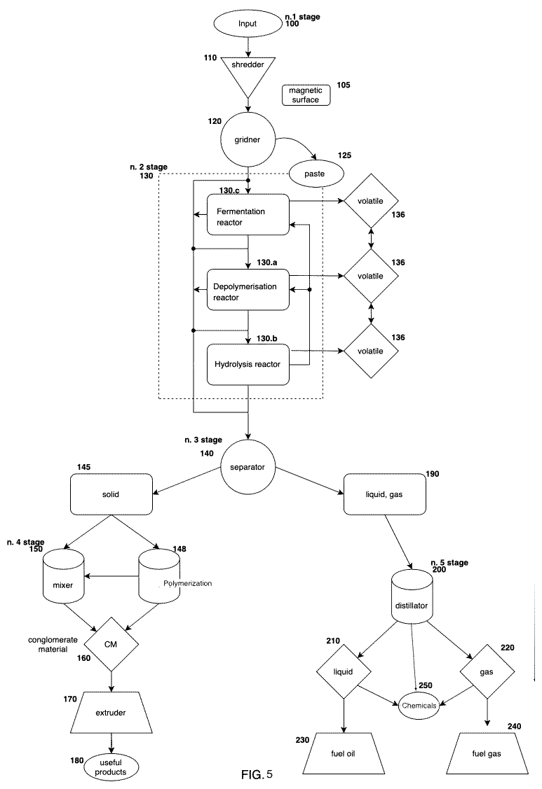

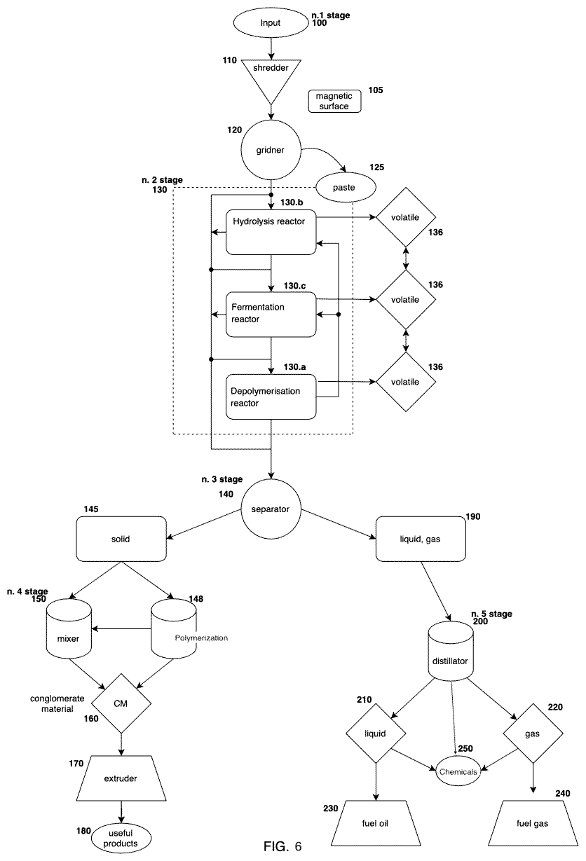

With reference to FIG. 1, the paste can be arbitrarily conveyed into a 130.b reactor for the decomposition/depolymerization of complex molecules by hydrolysis. The hydrolysis mechanism is particularly useful before subsequent steps of the process, which may be, for example, but not limited to, the 130.a pyrolysis, the 130.c fermentation or the separation of hydrolysis products in the third stage 140. In some embodiments of the present invention, knowing or assuming the percentage of input materials, it was provided with the implementation, in a special container, enzymatic hydrolysis thanks to cocktails of specific enzymes prior to step 130.c fermentation. In other embodiments of the present invention, knowing or assuming the percentage of the input materials in a special container, hydrolysis of mixed acid type has been implemented prior to further steps illustrated by the following invention. In still other embodiments of the present invention, knowing or assuming the percentage of the input materials in a special container, we have implemented thermal hydrolysis prior to further steps illustrated by the following invention. An ordinary skilled in the art, knowing, ignoring or assuming the percentage of input, is certainly able to understand the best type of hydrolysis to be implemented in relation to the quality of the final product and the consistency with further subsequent steps illustrated by this invention. The enormous advantage illustrated by this invention is a system of tubes and movable doors that, thanks to applied forces, is able to carry an input, of which the percentage of materials is unknown, known or hypothetical, in special chambers which are dedicated to the best process, be it chemical, biological, mechanical, physical or a combination of the above, in order to obtain in relation to the input material, final products of effective quality and economic and energy value. In FIG. 3 is illustrated the diagram representative of some embodiments of the present invention, where it can be inferred as the system of tubes and movable doors, which are represented respectively by directional arrows and knots applied to the latter, is able to transport the input material, in multiple chambers arranged in series, in parallel or without a precise configuration, each suitable for a different type of process. The advantage of the present invention, unlike others similar to it, is exemplified by the diagram in FIG. 3 representative of some embodiments of the present invention, since the system of pipes, mobile doors and applied forces are able to process the input material in the best way or in the best way, also implementing different combinations of processes, exploiting different chambers with different variables such as but not limited to, rooms with different temperatures, pressure, PH, percentage of water, solvents or additives, presence of biological forms such as bacteria of various nature and realm, presence of different types of enzymes, presence of volatile components such as oxygen, nitrogen, carbon dioxide, methane, ethane and variables with a dynamic relevance such as time, coenzymes, enzymes etc. Each room, therefore, can represent a different type of environment with different chemical, physical and biological as well as mechanical characteristics and variables, being in fact present in some embodiments of the invention, mechanical and/or physical process application machinery, specially inserted in the whole chamber represented by the second stage, such as but not limited to, rotating discs, mobile blades, sonic or ultrasonic or microwave devices, plasma devices, vibrational, rotational, translational, roto-translational, gravitational and similar devices of various kinds able to apply forces and to simplify, improve and speed up the process, in order to obtain the best products in relation to the input materials. Furthermore, in some embodiments of the present invention, in addition to multiple cameras suitable for the realization of the processes of the second stage, there are multiple chambers—with the same characteristics and variables such as, but not limited to, rooms with equal temperature, pressure, PH, percentage of water, solvents or additives, presence of biological forms such as bacteria of various nature and realm, presence of different types of enzymes, presence of volatile components such as oxygen, nitrogen, carbon dioxide, methane, ethane and time—that are arranged in series, in parallel or randomly compared to the other rooms of the second stage, this multiple chamber were found to be advantageous and wonderfully performing in order to achieve a dynamic process in its entirety favoring the reduction for example, but not limited to the waiting times of some processes and allowing the process as a whole to be competitive, scalable and sustainable, as defined in this invention, in relation to other similar processes. Moreover, in some embodiments of the present invention, another essential advantage was the ability to power multiple machines thanks to energy from renewable sources such as, but not limited to, solar energy, wind energy, Wave energy, geothermal or energy produced by biofuels and biogas extracted and obtained as useful products and high energy value, from some embodiments of the present invention. An ordinary skilled in the art, based on the percentage of input materials, although it may be known, unknown or hypothetical, is able to implement the best process or the combination of the best processes of the second stage using, for example, but not limited to pumps with applied forces, mobile doors, electronic devices such as computers and sensors and precise mechanical machinery such as those mentioned above and arbitrarily adjusting some variables such as, but not limited to, temperature, pressure, PH, percentage of water, solvents or additives, presence of biological forms such as bacteria of various nature and realm, presence of different types of enzymes, presence of volatile components such as oxygen, nitrogen, carbon dioxide, methane, ethane and arbitrarily change the time of the processes feasible in the second stage, in order to obtain an excellent process, able to achieve the desired product in relation to the percentage of input materials considered. Other alternative embodiments of this invention are depicted in FIG. 3, FIG. 4, FIG. 5, FIG. 6; from these representations, it is possible to deduce the different combinations feasible in the second stage. Moreover, they have been represented thanks to directional arrows and nodes, tubes with a specific direction, respectively, in concomitance to the applied forces, and mobile doors which can change, as mentioned above, their position thanks to manual or electronic valves connected to an operator and/or a computer, able to highlight the best path or paths feasible in relation to the input material considered. In the diagram in FIG. 3, for example, the possibility of using multiple chambers/reactors 130.a, 130.a.1, 130.a.2, 130.a.n in the second stage, each potentially able to accommodate different environments, as defined beforehand. Room 130.a.n represents the possibility of arbitrarily arranging multiple rooms. In the diagram in FIG. 3, for example, the possibility of using multiple chambers/reactors 130.b, 130.b.1, 130.b.2, 130.b.n the second stage, each potentially able to accommodate different environments, as defined beforehand. Room 130.b.n represents the possibility of arbitrarily arranging multiple rooms. In the diagram in FIG. 3, for example, the possibility of using multiple chambers/reactors 130.c, 130.c.1, 130.c.2, 130.c.n in the second stage, each potentially able to accommodate different environments, as defined previously. Room 130.c.n represents the possibility of arbitrarily arranging multiple rooms. In FIG. 4, it is possible to appreciate the possibility for some embodiments of the present invention, to modify the main configuration, we can deduce as 130.b and 130.a, they are arranged in a different configuration regarding the same ones in FIG. 1. This change of configuration can be deduced by an ordinary expert in the art, relating to a precise percentage of input materials, although it may be knowable, hypothetical or unknowable or to factors such as, but not limited to, seasonality, geographical factors, force majeure events, periodicity, change in macroeconomic trends, food scarcity, etc. which may affect the percentage of input materials, making it recognizable, hypothetical or unknowable or possibility of the need for some products compared to others. In FIG. 5, it is possible to appreciate the possibility for some embodiments of the present invention, to modify the main configuration, it can be deduced as 130.c, 130.b and 130.a, they are arranged in a different configuration regarding the same ones in FIG. 1. This change of configuration can be deduced by an ordinary expert in the art, relating to a precise percentage of input materials, although it may be knowable, hypothetical or unknowable or to factors such as but not limited to, seasonality, geographical factors, force majeure events, periodicity, change in macroeconomic trends, food scarcity, etc. which may affect the percentage of input materials, making it recognizable, hypothetical or unknowable or possibility of the need for some products compared to others. In FIG. 6, it is possible to appreciate the possibility for some embodiments of the present invention, to modify the main configuration, it can be deduced as 130.c, 130.b and 130.a, they are arranged in a different configuration regarding the same ones in FIG. 1. This change of configuration can be deduced by an ordinary expert in the art, relating to a precise percentage of input materials, although it may be knowable, hypothetical or unknowable or to factors such as but not limited to, seasonality, geographical factors, force majeure events, periodicity, change in macroeconomic trends, food scarcity, etc. which may affect the percentage of input materials, making it recognizable, hypothetical or unknowable or possibility of the need for some products compared to others. A representation of some embodiments of the present invention is represented in FIG. 2. In the first stage, input materials 100 are shredded with a shredding machine 300 and a grinder 310 in such a way as to transform the input materials into a paste 125. This paste is completely or partially stripped of metallic materials by means of one or more static and/or dynamic 105 magnetic surfaces. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.c.2. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.c.1. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.c.2. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.c.n. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.c. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.b. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.b.1. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.b.2. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.b.n. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.a. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.a.1. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.a.2. This paste, thanks to a system of hermetic tubes and applied forces, is transported, in the second stage 130, in this stage can be, optionally and in relation to the percentages of the input materials, conveyed inside a first reactor as an example, but not limited to, the reactor 130.a.n. This paste, thanks to a system of hermetic tubes and applied forces, is transported, in the second stage 130, in this stage can be, optionally and in relation to the percentages of the input materials, conveyed inside a first reactor or directly conveyed in the third stage 350/140. In FIG. 2, it is possible to appreciate how the paste 125 can be optionally conveyed inside a reactor used for hydrolysis 130.b. Keeping in mind the potentially heterogeneous nature of input materials and therefore of the 125 paste, it is possible that there may be traces of different materials such as, but not limited to, organic, inorganic, plastic, metallic, etc. Exemplary embodiments of the present invention included a type of thermal hydrolysis, which administered heat and pressure to the paste, has been shown to be able to break the more complex bonds of macromolecules that make up the paste and to obtain a mixture of monomers or simpler chains. The process of thermal hydrolysis, in the aforesaid embodiments of the present invention, has been carried out in a range of temperatures between the 50° C. and the 500° C. with a pressure between the 200 psig and the 900 psig. This process was carried out in one or several compartments within the hydrolysis chamber and provided for compression at high pressures for between 10 minutes and 150 minutes, followed by an environment with lower pressure in which the paste was conveyed for between 50 minutes and 400 minutes. This process took place in environments with a PH in a range between 4 and 8. Such a procedure is fully appreciable and understandably advantageous by an ordinary skilled in the art because the paste is sterilized and decomposed in an agglomeration of monomers and simple molecular chains that make the aforementioned paste more processable in further steps of the following invention. Some, not limiting, examples have predicted the above thermal hydrolysis for a period of time of about 50 minutes, in an environment with a pH of about 5, at a temperature of about 250° C. and with a significant pressure of around 750 psig, this procedure proved essential prior to the fermentation step 130.c, which in the present embodiments, considering the percentage of input materials, which in this case provided high percentages 70%-80% of organic materials with high lignin and cellulose contents, has been implemented thanks to microbial digestion. The latter procedure is fully appreciable and understandably advantageous by an ordinary skilled in the art, since the performance of microbial digestion in the fermentation reactor 130.c, has proven to be more performing than similar situations with disadvantageous characteristics. The temperature of about 250° C. has been adjusted to the degradation of lignin; it begins to degrade at temperatures even below 250° C. and has also been advantageous for cellulose, which has a degradation temperature very close to 250° C. and hemicellulose, which degrades from 120° C. Generally, the process of hydrolysis can include the breakdown of peptides into proteins and the consequent possibility of obtaining amino acids, triglycerides, fatty acids and sugars. In this process, fats are divided into fatty acids and glycerol, the degradation of lignocellulose occurs, and carbohydrates are usually broken down into simple sugars. As shown in FIG. 1, the hydrolysis process 130.a, the depolymerization and/or pyrolysis process 130.b and fermentation 130.c, can produce a mixture of gaseous and volatile substances, potentially harmful to man and the environment, such a mixture of substances, 136 containers are designed to withstand possible pressures and irritants. These volatile substances present in the containers can be stored or conveyed, thanks to a system of pipes and forces applied, directly to the fifth stage 200, where thanks to a system of distillers and condensers, which will be described in detail below, are divided into its components and transformed into useful products. In some embodiments of the present invention, the hydrolysis process 130.a, produced a large number of volatile substances such as carbon dioxide, ammonia, nitrogen and sulfuric acids, such volatile substances, once extracted and stored in special chambers, were conveyed, thanks to a system of pipes and applied forces, directly to the fifth stage 200, where thanks to a system of distillers and capacitors, were divided into its components and transformed into useful products 230, 240 and/or chemicals 250. In some embodiments of the present invention, the first hydrolysis step 130.b, took place within one or more hydrolysis reactor 130.b, 130.b.1, 130.b.2, 130.b.n, shown in FIG. 2, each one that in its turn contained one or multiple compartments and that I presented in the complex environments, as previously defined, different. Such environments could, for example, but are not limited to, present mechanical equipment capable of speeding up the process, such as mixing the paste while processed or shaking or compressing it. In some embodiments, the hydrolysis reactor contained three different compartments, connected by the mechanical opening of mobile doors, each with different physical, chemical, biological and mechanical variables such as, but not limited to, a temperature difference between the first and second compartments of more than 400° C., a pressure difference between the three compartments of about 400 psig and the presence of a rotating mechanical vibrating disc at the bottom of the first two compartments. In some embodiments of the present invention, the first hydrolysis step 130.b, took place inside the hydrolysis reactor 130.b., which contained a protic acid, which was able to catalyze chemical bonds and untie them into simpler bonds. Generally, hydrolysis of this type occurs thanks to the nucleophilic substitution reaction and the addition of water. This process has been revealed in some embodiments of this performant invention to treat lignocellulose and transform these molecules into fermentable sugars, fatty acids and peptides. In some embodiments of the present invention, the first hydrolysis step 130.b, took place inside the hydrolysis reactor 130.b. which contained high percentages of sulfuric acid, which was able to catalyze the chemical bonds and untie them into simpler bonds in an ambience of temperature between 40° C. and 70° C. and with a pH of about 5. In some embodiments of the present invention, the first hydrolysis step 130.b, took place inside the hydrolysis reactor 130.b.1. which contained high percentages of trifluoroacetic acid, which was able to catalyze the chemical bonds and untie them into simpler bonds in a temperature environment of between 50-50° C. and 70° C. In some embodiments of the present invention, the first hydrolysis step 130.b, took place inside the hydrolysis reactor 130.b.2. which contained high percentages of Formic acid, which was able to catalyze the chemical bonds and untie them into simpler bonds in a temperature environment of between 6° and 80° C. and with a PH of about 5. In some embodiments of this invention, the first step of hydrolysis 130.b, took place inside the hydrolysis reactor 130.b.n, which contained high percentages of nitric acid, which has been able to catalyze the chemical bonds and untie them into simpler bonds in a temperature environment of 55° C. In still other embodiments of the present invention, the paste 125 was conveyed in the second stage in a hydrolysis reactor 130.b, which presented an environment with specific characteristics suitable for splitting the more complex bonds of macromolecules, proteins have been transformed into amino acids, peptides and polypeptides thanks to the help of specific enzymes that have allowed to obtain a large abundance of amino acids of levorotatory shape. This process required a particular control of the environment, where a specific temperature, PH, addition of chemicals, water, additives etc., have been set. Some non-exhaustive examples concern the use of the hydrolysis reactor 130.b for the treatment of a paste whose content in about 85% of organic material was assumed mainly with macromolecules of lignocellulosic nature, in this case, the temperature of the environment was set in a range between 45° C. and 60° C., the addition of chemicals was omitted, and a quantity of water was added, using pumps and applied forces, such as to make the compound suitable for a type of enzymatic hydrolysis focused on cellulolytic enzymes and proteolytic enzymes, in a time between 10 and 14 hours. In some embodiments of the present invention, as illustrated in FIG. 2, the paste, after being conveyed into the hydrolysis reactor 130.b, for a period of about 12 hours, was conveyed directly to the third stage in a separator 350, such machinery, consisting of centrifuges and/or filters and/or condensers or a combination of the above, has been able to divide the liquid fraction obtained by the hydrolysis process from any sediment, part of the liquid fraction was vaporized in step 390 of the fifth stage and conducted by the divider in special chambers for 220 volatile substances before being further processed. The liquid part was conveyed to a 390 distiller and then mixed in 210 liquid substances chambers and then divided into 250 chemical substances that cannot be processed on site and substances suitable for the production of biofuel/oil fuel 230. The sediments and the remaining solid part of the paste after the hydrolysis process were conveyed in the fourth stage in a 365 polymerization and then in a 360 mixer to be processed in a conglomerate material 160 with the help of heat and mechanical forces applied. Other non-exhaustive examples concern the use of the hydrolysis reactor 130.b for the treatment of a paste whose content in about 60% of organic material was assumed, and a remaining part of about 40% including materials of a heterogeneous nature such as plastics, glass, ceramics, metals, fibers, etc. in which case the temperature of the environment was set over 80° C., the addition of chemicals was crucial to speed up the process, and a quantity of water was added to the compound of about 40% of the total weight, by means of pumps and applied forces, such as to make the compound suitable for a type of mixed enzymatic and chemical hydrolysis focused on cellulolytic enzymes and proteolytic enzymes as well as a mixture of chemicals understandable by an ordinary skilled in the art, in a time of more than 14 hours and with a neutral PH. Other non-exhaustive examples concern the use of the hydrolysis reactor 130.b for the treatment of a paste of which the content in about 50% of MSW was assumed, and a remaining part of about 50% including heterogeneous materials such as plastics, glass, ceramics, metals, fibres, organic etc., in this case, the temperature of the environment was set over 70° C., the addition of chemicals has been fundamental to speed up the process, and a quantity of water has been added to the compound of about 40% of the total weight, with the help of pumps and applied forces, such as to make the compound suitable for a type of mixed enzymatic and chemical hydrolysis focused on Digestive enzymes of the type Lipases and Amylases, as well as a mixture of chemicals understandable by an ordinary expert in the art, in more than 10 hours. In some embodiments of the present invention, as illustrated in FIG. 2, the paste, after being conveyed into the hydrolysis reactor 130.b, for a period of about 10 hours, was conveyed to a suitable fermentation reactor 130.c, which thanks to precise processes were able to further process the paste, in order to obtain higher quality products, these fermentation products, both in liquid and solid and gaseous form, were conveyed to the third stage in a 350 separator, this machinery, consisting of centrifuges and/or filters and/or condensers or a combination of the above, has been capable of dividing the liquid fraction obtained by the fermentation process from any sediment, part of the liquid fraction was vaporized in step 390 of the fifth stage and conducted by the divider in special chambers for 220 volatile substances before being further processed. The liquid part was conveyed to a 390 distiller and then conveyed to 210 liquid chambers and then divided into 250 chemical substances that could not be processed on site and substances suitable for the production of biofuel/oil fuel 230. The sediment and the remaining solid part of the dough after the fermentation process was conveyed in the fourth stage in a mixer 360 to be processed in a conglomerate material 160 with the help of heat and mechanical forces applied.

### Second Stage: Decomposition by Fermentation

Exemplary embodiments of the present invention, as illustrated in FIG. 2, the paste 125, after step 310 in which it was mechanically decomposed, was conveyed into the fermentation reactor 130.c, this reactor had useful characteristics to process the paste before the third stage of separation 350. The useful characteristics concern specific environments or the set of characteristics and physical, chemical, biological, mechanical or combinations of the previous, the whole of a given chamber or more chambers or reactors of the second fermentation stage such as but not limited to, rooms with a certain temperature, pressure, PH, percentage of water, solvents or additives, presence of biological forms such as bacteria of various nature and kingdom, presence of fungi, presence of different types of enzymes, presence of volatile components such as oxygen, nitrogen, carbon dioxide, methane, ethane etc. The fermentation process is highly performing for the process of this invention in the treatment of potentially heterogeneous materials. It has been amply demonstrated that the fermentation process, used for millennia by human beings, is essential in the treatment of numerous types of materials and substances. Fermentation, thanks to the ability to oxidize and reduce the same molecule, is remarkably suitable to treat materials such as carbohydrates, molecules highly present in treatment situations of heterogeneous input materials. In some embodiments of the present invention, fermentation was carried out in the absence of oxygen, while in other embodiments, it was implemented in an environment with a mixture of sulfates, nitrates, etc., including oxygen. In some embodiments of the present invention, the fermentation process took place in the whole of a chamber, where the dough was conveyed, in which there was an environment that had low oxygen levels and a high percentage of microorganisms of the Saccharomyces type. This last chamber, thanks to the aid of specific microorganisms and fungi such as, but not limited to, the Saccharomyces cerevisiae has processed the paste in an excellent way creating a large amount of ethanol. This process has been divided into several phases, the main ones being the splitting of complex sugars into simple sugars and the consequent formation of ethanol. Fermentation took place by means of enzymes such as the invertase enzyme, which split complex sugars such as sucrose into simpler substances such as fructose and glucose. In the cytoplasm of the organism under consideration, glycolysis has been verified thanks to environmental variables kept under control, by means of specific sensors, indicators, etc., such as the quantity of oxygen, the temperature, the PH etc. In other embodiments of the present invention, the fermentation process took place in the whole of a chamber, in which the dough was conveyed, in which there was an environment with low oxygen levels and a high percentage of microorganisms of the lactobacilli and fungus type. This last chamber, thanks to the aid of specific microorganisms such as, for example, but not limited to, the Lactobacillus, Lactococcus, Streptococcus, Leuconostoc, Weissella, Oenococcus, Pediococcus, Carnobacterium, Enterococcus, L. rumins, L. acidophilus, etc. has processed the paste excellently creating a large amount of lactic acid. This process has been divided into several phases, the main ones being the splitting of fermentable sugars into pyruvic acid molecules and the consequent formation of lactic acid. Fermentation took place thanks to environmental variables kept under control, by means of specific sensors, indicators, etc., such as a very low if not absent quantity of oxygen, temperature, PH etc. In some embodiments of the present invention, the fermentation process took place in the whole of a chamber, where the dough was conveyed, in which there was an environment that had low oxygen levels and a high percentage of microorganisms of the Lb. Casei type, Lb. Paracasei, Lb. Curvatus, Lb. Pentosus, Lb. Plantarum, Lb. Saki, Lb. Rhamnosus, Lb. Bavaricus. Etc. and fungi. The latter chamber processed the paste excellently, creating a large amount of ethanol, lactic acid and volatile substances. This process has been divided into several phases, the main ones being the splitting of complex sugars into simple sugars and the consequent formation of ethanol, carbon dioxide and lactic acid. Fermentation took place by enzymes such as the invertase enzyme, which split complex sugars such as sucrose into simpler substances such as fructose and glucose. In the cytoplasm of the organism under consideration, the oxidation of glucose-6-phosphate to 6-phosphogluconate occurred, which was subsequently decarboxylated, producing sugar and carbon. This sugar was then transformed into glyceraldehyde-3-phosphate and acetyl phosphate, which was transformed into lactic acid and ethanol. This process took place thanks to environmental variables kept under control, by means of specific sensors, indicators, etc., such as the amount of oxygen, the temperature, the PH etc. In still other embodiments of the present invention, the fermentation process took place in the whole of a chamber, in which the dough was conveyed, in which there was an environment that had low oxygen levels and a high percentage of microorganisms and fungi of the type belonging to Enterobacteria such as E. Coli. Etc. The latter chamber has excellently processed the paste creating a large amount of ethanol, lactic acid, acetic, succinic, formic CO2, hydrogen and other volatile substances. This process has been divided into several phases, the main ones being the splitting of glucose into a series of products such as acetylcylate, malat, D-lactat. Fermentation took place thanks to different types of enzymes and coenzymes and through different types of reactions such as phosphoenolpyruvate carboxylation etc. This process took place thanks to environmental variables kept under control, by means of specific sensors, indicators etc., such as the amount of oxygen, the temperature, the PH etc. In further embodiments of the present invention, the fermentative process was held in a particular environment. This environment had a very high component of bacteria such as, but not limited to, Bacillus Megaterium, Rhodococcus, Ralstonia metallidurans, Pseudomonas, Wautersia eutropha, etc. which, through the fermentation of sugars and lipids, have produced useful substances, such as some polyesters polymers that are linear macromolecules, such as, but not limited to, Polyhydroxyalkanoate PHA, Polyhydroxybutyrate PHB, Poly(hydroxybutyrate-hydroxyvalerate) PHB/HV, Poly(e-caprolactone) PCL, etc. The production of polyesters polymers in the fermentation stage is advantageous for the creation of useful products such as a conglomerate material with better mechanical characteristics and optimal fuel gas and fuel oil products compared to the same amount of heterogeneous material input processed and treated differently. These properties will be described in detail below. The production of these types of products has taken place thanks to bacterial cultures developed in particular environments and with controlled characteristics such as, but not limited to, percentages of nitrogen, phosphorus, sulfur, PH, temperature etc. This process took place thanks to the absence of certain nutrients such as nitrogen and oxygen, which encouraged bacteria to accumulate such linear macromolecules as a carbon source in reserve. An ordinary skilled in the art is able to understand which is the best type of fermentation or the best types of fermentation and the various possible combinations in the second stage in order to obtain the desired output products in relation to the percentage composition of the input products. There are various industrial processes for some of the examples shown; some embodiments include a batch/Fed-batch technique in which the paste remains to ferment with less direct control by an operator of the process dynamics; this process has some disadvantages, such as the continuous sterilization of environments, tubes etc. such a batch process may require the addition of ingredients, chemicals, for example, to limit the formation of foam by regulating the pH of the environment. The different stages of batch fermentation can take place in one or several chambers, connected with a system of pipes and applied forces. In further embodiments of the present invention, the fermentation process was held in a particular environment with the presence of mixed cultures of bacteria, even if naturally evolved, thus more resistant to the contamination of possible pathogens and harmful agents. Temperature regulation was an important factor in the process, which involved, for example, thermophilic bacteria with the consequent production of lactic acid at temperatures of about 60° C. Other environments have provided examples with a high presence of salt, an environment consistent with the growth of certain halophilic bacteria that have been useful in order to produce bioplastics. In still other embodiments, fermentation has been accelerated and optimized thanks to a system of tubes, plug flow reactors, etc., able to change the number of substrates and products and recycle cells between the entrance and exit of the fermentation reactor.

### Third Stage: Separation (Solid, Liquid, Gas)

In relation to FIG. 1, the paste, after being processed in the second stage by the reactor 130.a and/or 130.b and/or 130.c, is conveyed in the third stage to a 140 machinery used for the separation of solids, liquids and gases. Such separation may, for example, involve the separation of solid substances such as minerals, solid residues of processes, plastics, ceramics, metals, glass and other solid or semi-solid substances that were present in the input materials 100 and that have not undergone substantial differences from previous processes. The separation of liquids includes main water, hydrocarbon liquids, oils, substances and mixtures produced by the steps of the second stage. It is possible that in this step, some divided products, such as water, are reintroduced in some steps of the process. It is also possible that separation methods may be extended for up to three days. A non-restrictive example concerns a mass of solids that have been dried to completely deprive them of liquid substances for a time longer than 48 h by optionally supplying jets of heat and/or introducing this solid mass in appropriately heated chambers of various shapes and sizes and with different mechanical characteristics. A non-restrictive example concerns a mass of solids introduced for more than 72 h in a chamber heated by concave and convex lenses, capable of heating the airtight environment and evaporating liquids, subsequently placed in rooms suitable for the storage of liquids 190 and then in the fifth stage 200. The separation step 140 can also provide multiple machines arranged in series or in parallel. In exemplary embodiments, the processed paste is transported initially in a volatile substance separator and then in a liquid/solid separator; then the split liquids are transported in a liquid/liquid separator, which are finally conveyed, by means of pumps and applied forces, in the storage stage 190. In other embodiments, the processed paste is transported initially in a volatile substance separator and then in a liquid/solid separator first; then it is transported in another separator, where the liquids are further separated from possible, solid residues, which have not been detected by the first separator, in addition, the split liquids are transported in one or more liquid/liquid separators, which are finally conveyed, by means of pumps and applied forces, in the storage stage 190. The separation methods of the third stage 140 can be numerous and of different shapes and sizes and with different environmental characteristics. Some embodiments involved the aid of multiple centrifuges, condensers, distillers, gravity-exploiting filters and separators capable of dividing not only substances in relation to their physical state but also in relation to their chemical and mechanical properties as a different boiling point, density, viscosity, different temperature in the physical state passages etc. and also the separation methods can happen thanks to the possible application of heat and pressure, such as the possible drying of the solid material after the subdivision, in some embodiments, it is provided with the administration of heat and pressure to the mixture of liquids during the separation step 140, in order to divide some liquids from the others. This presence of multiple separators able to divide solid substances from gaseous and liquid ones represents a fundamental stage of the process in its entirety, and thanks to the help of many machines, even with the possibility of processing the same substances more than once, it is an essential benefit of the present invention and symbolically represents a key step in the objective of the following invention of replicating the animal digestive system.

### Fourth Stage: The Conglomerate/Composite Material

In relation to FIG. 1, the solid materials 145 that have been appropriately divided by the separator 140, can be, based on the percentage of input materials 100 and in relation to the steps made in the second stage, conveyed in a 150 mixer or in an apparatus suitable for curing 148. An ordinary skilled in the art is able in an arbitrary way to subdivide the solids suitable for the polymerization process with respect to those directly conveyed in the mixer based on the previous processes of the second and third stage and in relation to the percentage of input materials 100. The curing process 148 in FIG. 1 or 365 in FIG. 2, which may be chain or stage type and may require one or several chambers arranged in series or in parallel, results to be of benefit and advantage in the process in its totality as it is able to transform the monomers and simple molecules that may have been produced by some steps of the second stage, in polymer chains. This chemical reaction is performing because it is capable of making materials with mechanical characteristics, optimal physical, chemical and biological and directly conveyable in step 160 of storage of conglomerate materials or in mixer 360 in FIG. 2 or 150 in FIG. 1 to be further processed or mixed with other types of solids. In some embodiments of the present invention, the conversion of monomers and other simple molecules included multiple manufacturing processes such as the addition of additives and substances, calendering, coating, and modelling of materials in specific shapes for the following steps as well as possible thermal modelling such as compression, injection etc. These steps were found to be a key factor before proceeding with further steps such as mixing 150, storage of any 160 conglomerate materials and subsequent step 170 extruder with subsequent shaping of 180 output useful products. In some embodiments of the present invention, the conversion of monomers in polymers require an appropriated softened state, required the administration of heat and subsequently a cyclic or batch polymerization process or, in some cases, a continuous process in which there was a better use of energy. The energy required or enthalpy to increase the unit mass of the polymer from the ambient temperature to that of processing is a characteristic known by an ordinary skilled in the art. The enthalpy of the process depends mainly on the structure of materials such as thermoplastics; for example, thermoplastics have larger enthalpies due to their amorphous counterparts. Due to the susceptibility of some polymers to thermal decomposition, in some cases, it is provided with a rapid administration of heat, resulting in rapid cooling of the mass. In other embodiments of the present invention, an adjustment of the viscosity of the solid mass was implemented by regulating temperature, pressure, molecular size and adding lubricants or chemicals to achieve a low viscous mass and directly conveyable in a 150 mixer in a storage chamber for 160 conglomerate materials and finally in a 170 extruder. The extrusion process is a process in which materials, which can be, for example, but not limited to, polymers, in the form of granules, powder, melt etc., are converted into products with predetermined sections. This process is allowed by administering heat and pressure to the input materials, forcing them out of an orifice and subsequently cooling or solidifying them with chemical reactions in order to obtain desired products. In still other embodiments of the present invention, a type of polymerization in solution has been implemented, where solids are inserted in a chamber containing different types of solvents such as hydrocarbon solvents. In further embodiments of the present invention, there is a type of polymerization in emulsion, in which there is a surfactant product in a chamber containing solids and a part of water. This process, together with the process in solution, has some advantages such as a reduced viscosity of the material and is performing thanks to the characteristics of the initiator soluble in water, unlike the monomers constituting the solid mass. The production of a large number of micelles that stabilize the droplets of monomer acting as reactors. The number of micelles formed depends on the amount of added surfactant that is often greater than the critical micellar concentration. In relation to FIG. 1, the solid materials 145 that have been appropriately divided by the separator 140 can be, based on the percentage of input materials 100 and in relation to the steps made in the second stage, conveyed in a mixer 150. An ordinary skilled in the art is able in an arbitrary way to subdivide the solids suitable for the polymerization process with respect to those directly conveyed in the mixer based on the previous processes of the second and third stage and in relation to the percentage of input materials 100. The mixing phase 150 can be formed by one or more mixers arranged in series or in parallel. In some embodiments of the present invention, the type of mixer was very similar to the well-known Z blade mixer. Such a mixer is useful for levelling solid or semi-solid materials. This machine consists of a U-shaped tank with two Z-shaped blades and each wheel in the opposite direction to the other for correct mixing of the material. This machine in some embodiments had a capacity of about 500 kg while in other embodiments of about 5000 kg to reach even 10000 kg. Some embodiments had a Z blade mixer composed of stainless steel and used for the administration of heat and/or pressure the solid or semi-solid mass resulting in its cooling. At the whole of such Z blade mixer in some embodiments, there are tangential and/or transverse blades designed for uniform mixing. In other embodiments of the present invention, solid-solid mixing took place by means of a foundry mulling process with sand molding. Such bentonite clay sand or fine coal dust and water have been mixed with other heterogeneous materials such as, but not limited to plastics, organic materials, inorganic etc. and finally shaped and cooled in the following stages 160 and 170 in order to obtain usable solid products 180. In still other embodiments of the present invention, the solid-solid mixing has happened through the addition of chemical substances like stabilizers, plasticized, dyes, deodorants etc., with the objective to increase the machinability, appearance and quality of the final product 180. In some cases, it is provided with a mixture of different types of plastics that have been melted, along with other types of heterogeneous materials, with the application of heat and/or pressure. Some non-limitative example regards mixing various kinds of molten plastic of the type PET, HDPE, PVC, LDPE, PP, PS, etc. and other heterogeneous materials included in the initial input 100 which have been heated to temperatures above 90° C. and have produced a composite material 160 with thermoplastic characteristics and high quality in terms of physical properties, chemical, biological and mechanical that was subsequently conveyed in an extruder 170 suitable for the modelling of this material conglomerate in usable products 180 and salable in the market. In further embodiments of the present invention, the mixer type 150 was contracted with an existing company and has not been modified in any way. In other embodiments of the present invention, the mixer type was contracted with an existing company but modified at will to obtain some desired characteristics. Some non-exhaustive examples relate to the use di un heating mixer with an induction heating system able to mix, heat, react, kneading, granulate, cooling, dry, etc. The utilization of that mixer is absolutely advantageous in respect to other type because the preparation of the conglomerate material is without dead zones in the process chamber, there are no shaft passages in contact with the product, the mixer is able to processing a lot of different materials also with extremely viscoplastic and consistency and also the inductive heating of the mixing pan enables to direct generation of heat in the wall of the mixing pan and this is advantageous because there is no heat lost due to heat transfer between heating medium and pan wall, and also the heat is homogeneous with a controllable temperature field in the area of the induction coils, also there is a good dynamic control behavior for optimum temperature control and reproducibility, a good and fast heat transfer between moved material and rotating pan wall by high temperature gradients and high surface-related power input and also a process control design free pre-selectable for the optimum temperature curves and also a great minimization of space requirement thanks to a heating unit integrated in the machine housing. That mixer enables an ordinary skilled in the art to control the speed of the mixing tool, from slow to fast and enable an optimally controllable preparation effect by using contact heat transmission in the plastic phase as well as in the mechanical generation of fluid. The mixer of the example also is able to work with a heating power between 5 kW and 300 kW and is able to mix the mass with a melted binder like the type of resin instead of binder solution with great consistency in regard to the porosity of the conglomerate material 160. Also, that mixer is able to heat solid/solid mixes and solid/liquid mixes to temperatures between 30° C. and 350° C. and to control an eventual evaporation process with an apposite apparatus. Also, that mixer is able to applied exothermic or endothermic reactions, control the phase separation of multiphase mixes with simultaneous drying under defined temperature and pressure and use a rotary evaporator. The composite material 160 is clearly linked to the input materials, which, as previously described, can be of different types. We therefore reiterate that this material may contain some heterogeneous materials of the industrial type waste, agricultural waste, municipal waste, commercial waste, Sewage sludge, Radioactive waste, Medical waste, paper, glass, metal, plastic (PETE, HDPE, polypropylene, PET, PP, LDPE, PVC, PS, polystyrene, film products, etc.), electronics, organics (food, leaves, grass, pruning, trimmings, branches, stumps, manures, lumber, wood, pallets, crates, agricultural waste, forest waste, etc.), inert material (piece of building foundation, paving, blocks, asphalt paving, asphalt roofing, gypsum board, rocks, drywall, carpet, soil, fines, stones, sand, brick, ceramics, fiberglass, mixed demolition debris, paint, auto batteries, lubricating oil, batteries, gas cylinder), Hazardous waste (pills, liquid creams and pharmaceuticals, household hazardous, pesticides, LED lamps, mercury containing items, vehicles and equipment fluids, caustic cleaners, fluorescent lamps), special waste (tires, bulky items, mattresses and foundations, ash, auto bodies, medical waste), miscellaneous (organic textiles, synthetic textile, shoes, purses, belts, solar panel, diapers, sanitary products, garden hoses, cigarette butts, cosmetics, straw basket, animal carcasses, mixed residue, shredding residue, feces, rubber sports balls, MRF residual fines, kitchen ceramics, synthetic rubber products). The conglomerate material 160 is often composed of mixing or glueing two or more materials. One material or class of materials constitutes the matrix, which is identified as a binder; the other materials are the filler, which can act as a reinforcement, with the intent to improve the rigidity, the resistance, etc. in the case of structural/construction material or other properties of the conglomerate material 160 like resistance to flame, resistance to the impact, abrasion, electrical conductivity, malleability, elasticity, plasticity etc. The matrix of the conglomerate material 160 can be fundamentally made of metallic, ceramic or polymeric materials. The filler can consist of synthetic fibres, particles, glass, short fibre, organic fiber etc. The conglomerate material 160 can have a reinforcement consisting of long fibres of materials such carbon, glass, Kevlar, metal etc. These types of elements in the conglomerate material are able to improve some properties of that who are strictly related with the less presence of a defect in the crystal molecular structure. The conglomerate material 160 can optionally be covered with homogeneous or heterogeneous materials. In order to obtain the desired conglomerate material 160 or the optimum conglomerate material in relation to the application area is mandatory for an ordinary skilled in the art to examine the most performant combination or type of process to consider in the rest of the total process of the present invention. In exemplary embodiments of the present invention, 145 solid materials, before being conveyed into the mixing process, presented a high percentage of bio-based polymers such as, for example, but not limited to, bioplastics types such as biobased PE, PET, PA, PTT and types of bio-based and biodegradable plastics such as PLA, PHA, PBS, starch blends but also types of biodegradable but fossil-based plastics such as PBAT and PCL. These types of plastics were either an integral part of the 100 input materials or were produced by one or more processes of the first, second and third stages. Such types of biobased or biodegradable plastics, different from conventional, have been found to be partially or totally suitable for a type of mixing with other topologies of materials, including conventional plastics and going to increase the characteristics and properties of the final conglomerate material and therefore his quality. A non-limiting example concerns the use of a series of PHA-based polymers, which have shown great miscibility with other plastics such as PVC, improving performance and its mechanical characteristics. The production and use of biodegradable or biobased polymers such as cellulose, chitin, starch, PHAs, polylactide, poly(e-caprolactone), collagen and other polypeptides appears to be necessary for the plastic industry at present and in the future in terms of sustainability. Such polymers are synthesized or formed in natural environments during the growth cycles of organisms. Some microorganisms and enzymes capable of processing certain polymers have been identified and potentially introduced into specific reactors of the second stage 130, constituting a fundamental initial step for the subsequent processing of these polymers. Generally, natural or biodegradable polymers are produced by biodegradation reactions as typically enzyme-catalyzed and occur in aqueous environments. Natural macromolecules contain hydrolyzable linkages such as protein, cellulose and starch a are often susceptible to biodegradation by hydrolytic enzymes of microorganisms. In some embodiments of the present invention, 145 biodegradable polymers were found among the solid materials: cellulose, lignin, starch, chitin, collagen, dextran, heparin, xanthan, hyaluronic acid, gelatin, elastin, fibrin, pectin and polyhydroxyalkanoates polymers belonging to the class of agro-polymers and biopolyesters such as polysaccharides, proteins, lipids, polyester produced by microorganism or plant, polyesters synthesized from bio-derived monomers, miscellaneous polymers, aliphatic Polyesters, aromatic Polyesters, polyvinyl alcohols and modified polyolefins. In exemplary embodiments of the present invention, some types of bioplastics were produced thanks to the third stage 130, especially in chambers suitable for fermentation or containing special types of bacteria 130.c, 130.c.1, 130.c.2, 130.c.n, and then conveyed to a curing chamber 148, an ordinary skilled in the art based on the percentage of input materials is able to determine which is the best process or the combination of processes suitable for producing bioplastics. Some non-exhaustive examples concern an input 100 composed of more than 90% agricultural waste and the consequent production of intracellular biopolymers phas, produced by bacterial fermentation of sugars and lipids occurred in the fermentation reactor 130.c.2, with a suitable environment for the production of phases and therefore regulated with specific variables such as temperature, PH, oxygen etc. moreover this environment had a very high component of bacteria such as, but not limited to, Bacillus Megaterium, Rhodococcus, Ralstonia metallidurans, Pseudomonas, Wautersia eutropha. The first step of the above fermentation was the process of inoculation, where the bacteria multiplied in an aqueous environment balanced with nutrition supply and air under optimum physical conditions. The next step concerned the PHA sinter under conditions not suitable for the growth and multiplication of bacteria and a relative oversupply of carbon. Some general characteristics of phass are water-insoluble and relatively resistant to hydrolytic degradation, a good ultraviolet resistance, biocompatibility with the medical application, a non-toxicity and less sticky than traditional polymers when melted. These characteristics make PHA and, in general, bio-based polymers an excellent product or an excellent combination with other types of materials in order to obtain a material 160 conglomerate with high mechanical characteristics such as strength and energy absorbance. Depending on the input materials 100 and the possible application of one or more steps of the second stage 130, the conglomerate material can have different properties to be suitable for various types of application. The following table shows some non-exhaustive examples and properties that have been found in the 160 conglomerate materials and some types of applications for which this material can be exploited. We reiterate how an ordinary skilled in the art can be able to understand, if necessary, the type or types of processes possible in the second stage 130 in order to obtain the best material conglomerate 160—also in relation to its practical application—based on the percentage of heterogeneous input materials 100.

TABLE 3

| N. | Properties | Application |
|---|---|---|
| 1 | Proportional | Springs, precision instrument |
| | limit | |
| 2 | plasticity | Forming, shaping, extruding, ornamental, |
| | | stamping, rolling, pressing |
| 3 | malleability | Rolling, hammering |
| 4 | toughness | Shock and impact loads |
| 5 | creep | Boilers, turbines |
| 6 | resilience | springs |
| 7 | hardness | Resistance to deformation, wear |
| 8 | fatigue | High-speed aero and engine turbine |
| 9 | elasticity | Materials used in tools and machines |

Instead, the following table 3 shows some non-exhaustive examples of details required for some types of concrete application 160. These possibilities of application and their relationship with the types of necessary characteristics that the concrete must have to be suitable for use, are of great help for an ordinary expert in the art to deduce the type or types, if necessary, processes in the second stage 130 and the possible addition of chemicals, additives, plasticizers, etc. in the mixer 150.

TABLE 4

| Application | | Requirements | |
|---|---|---|---|
| Area | Examples | Area | Requirement Details |
| Construction | Building, | Stability, Fire | Mechanical resistance to static |
| | bricks, | Security | and dynamic actions<br> No |
| | pipeline, | | emission of harmful substances |
| | floating | | |
| | structures | | |
| Home | Household | Toxicity, Fire | No emission of harmful |
| Application | objects, | Security, Odor, | substances<br> Acoustic |
| | everyday | Touch-sensitivity, | insulation<br> Control of thermal |
| | materials, | Visual Impact, | inertia<br> Air tightness<br> Solar |
| | textiles | Modelling, | factor<br> Absence of odor |
| | | Geothermal, Ease | emission<br> Resistance to |
| | | of Use and | dust<br> Image quality |
| | | Operation | |
| Industrial/House | Insulating | Stability, Fire | Mechanical resistance to static |
| Component | materials, | Security, Insulation | and dynamic actions<br> No |
| | fireproof, | | emission of harmful |
| | acoustic, | | substances<br> Control of thermal |
| | waterproof | | inertia<br> Solar factor |

The following non-exhaustive table 4 shows a series of additives that can be added in step 150, step 145, step 160, step 170 or, if necessary, in one of the processes of the second stage 130. An ordinary expert in the art can easily deduce the best additives to be used in relation to the quality of the final product and its type of application. Table 5

| | Typical | | |
|---|---|---|---|
| | concentration | | |
| Additive type | % | Description | example |
| Flame | 1-35 | Adapt for petrochemical | Brominated flame |
| retardans | | plastic in retarding flame | retardants, chlorinated |
| | | | paraffins |
| Heat | 0.2-7 | Prevents heat related | Lead, cadmium, barium, |
| stabilizers | | degradation | zinc, calcium stearate |
| Fillers | 0-60 | Changes appearance | Calcium carbonate, talc, |
| | | and mechanical | glass, carbon black, |
| | | properties | carbon fibre |
| Plasticizers | 5-70 | More durability and | Phthalates, adipate |
| | | flexibility | esters, citrate esters |
| Impact | 10-45 | Improve: toughness, | Chlorinated |
| modifiers | | resistance, etc. | polyethylene |
| Colourants | 0.001-10 | Imparts colour | pigments |
| Antioxidants | 0.05-5 | Protect against | Phenols, phosphite |
| | | degradation | esters, thioethers |
| Lubricants | 0.1-3 | Assists in molding the | Paraffin wax, wax |
| | | plastic | esters, zinc, long-chain |
| | | | fatty acid amides |
| Light | 0.05-4 | Protect against UV | HALS, UV blockers |
| stabilizers | | damage | |
| Other | | Various | Antimicrobias, |
| | | | antistatic, etc. |

The conglomerate material 160 eventually reduced to powder, granules, pellets etc., is, after the mixing step 150, conveyed in an extruder 170. The extrusion process 170 can be implemented in multiple extruders arranged in series or in parallel. The extrusion process is a process capable of deforming the conglomerate material resulting in the production of desired parts/objects and optionally constant section; some non-restrictive examples include the production of utility objects such as pipes, bars, slabs, etc. the extrusion process consists in forcing the concrete to pass a shape that reproduces the external shape of the desired workpiece. At the exit of the template, the material is cooled or in some cases, subjected to further processes such as vulcanization. In exemplary embodiments of the present invention, there were multiple extruders of which, some of the typologies of pulling-extrusion that allow obtaining plastic profiles reinforced by fibers and different substances. In other embodiments of the present invention, there were multiple extruders of which some of the type blow molding, able to produce plastic containers as jars, bottles, jars etc. In the latter process of blow molding or blow molding, the extruded hose is surrounded by a mold and blown with compressed air against the inner wall of the mold. In still other embodiments of the present invention, there were multiple extruders of which some of the type coextrusion able to handle different materials obtaining, for example, gaskets with a rigid core and a softer and hermetic outer part. In some embodiments that had multiple extruders, there were some purchased from a manufacturer, other products by hand according to need and others purchased previous contractualization of some structural changes. In some embodiments, the extrusion of the hot type was carried out, in others of the cold type, in others of the friction type.

### Fifth Stage: Distiller

In FIG. 1, during the separation process 140, as mentioned above, it is possible that the mixture of liquids and gas contained in one or more storage chambers 190 is previously divided by the separator 140. In some embodiments, such subdivision has concerned the separation of mixed liquids from the water thanks to different processes such as dehydration via depressurization, heat administration, vaporization, etc. some mixtures containing hydrocarbon liquids and water have undergone one or more separations using, for example, a centrifuge suitable for the separation of liquids/liquids, a distillation column, a condenser, a gravitational separation column etc. The percentage of water extracted from the separation process 140 is optionally re-circulated between input 100 materials and very often includes various dissolved substances such as amino acids, potential proteins, monomers, organic molecules, granules of heterogeneous substances, coal, microplastics, etc. these substances can optionally be divided into step 140 or into step 200 distillation. The 200 distillation step in FIG. 1 from the beginning to the fifth stage of the process this step may include one or more series or parallel distillers or other separators suitable for obtaining the desired products. Distillation is a technique capable of separating one or more substances present in a mixture by exploiting the difference in the boiling points of these substances and, therefore, their volatility difference. The distillation step can separate complex mixtures and have as output more different mixtures with different compositions, which can be conveyed in the following steps 210, 220 or 250 depending on the type of chemical composition. The distillation step may also be necessary to purify individual substances prior to their deposit in steps 210, 220 or 250. Any waste substances deposited in a special airtight chamber 250 destined for further procedures or reinserted, according to the characteristics and properties, in some steps of the following invention. In exemplary embodiments of the present invention, there are distillation columns consisting of three fundamental parts optionally combined with other parts necessary to obtain efficient output products. The three fundamental parts concern a cylindrical column, a boiler and a condenser. In some embodiments of the present invention, there was a plate-type distillation column in which there is a contact between the liquid current and the gaseous current at equidistant horizontal surfaces with overflows for the passage of the liquid current and holes as well as fixed valves, mobile valves and bells for the passage of the gas current. In other embodiments of the present invention, there was a distillation column of the filling type in which the contact between the liquid current and the gaseous current occurs at the surface of the blocks or filling bodies contained in the column; such bodies are generally made of inert material to the treated substances. The principle of operation of these distillation columns is based on the presence of a liquid phase and a gaseous phase that generally come into contact with the counter current and a boiler and a condenser capable of taking the desired substances. Various techniques can be used in the process and an ordinary skilled in the art can easily deduce which are the best techniques to be used in the 200 stage in relation to 100 input materials and the desired products such as fuel oil 230, fuel gas 240 and any chemical substances 250 not processable on-site but potentially with a not negligible economic value. Some non-exhaustive examples of techniques adopted in the 200 distillation process concern the type of flash distillation with appropriate chambers for the adoption of this type arranged in series or in parallel, a type of continuous or batch distillation with appropriate chambers for the adoption of this type arranged, if necessary, in series or in parallel, a type of distillation with reflux with appropriate rooms for the adoption of this type arranged in series or in parallel, a fractional distillation with appropriate rooms for the adoption of this type arranged in series or in parallel, azeotropic distillation with suitable chambers for the adoption of this typology arranged in series or in parallel, steam distillation with suitable chambers for the adoption of this type arranged in series or in parallel, an extraction distillation with appropriate chambers for the adoption of this type arranged in series or in parallel, a molecular distillation with appropriate chambers for the adoption of this type arranged in series or in parallel, a reactive distillation with appropriate chambers for the adoption of this type arranged in series or in parallel, etc. A performance distillation process 200 is, therefore, able to separate gases such as hydrogen, methane and ethane from liquids and mixtures as hydrocarbon liquids. In exemplary embodiments of the present invention, a fractional distillation technique has been applied that can bring hydrocarbon liquid to a boil at a temperature of about 100° C. and extract various gases including ethane, methane and hydrogen. Part of the gases potentially adaptable to become quality fuel gas products are conveyed to one or more 240 depots. The hydrocarbons suitable to become fuel oil performing were conveyed in appropriate chambers for the deposit 230 while the remaining components, potentially of high economic value, have undergone other distillation processes 200 and finally conveyed in one or more 250 chambers or optionally reinserted in one or more steps of the process. To minimize the risk of explosions and release of potentially toxic substances to animals or the environment in general, all distillers, chambers etc. of the fifth stage have high levels of tightness and numerous safety devices and sensors able to control the process variables as well as walls corrosion and resistant to chemicals present. It is also possible that one or more distillation columns are buried or semi-buried to minimize any risk of an accident. Some non-limiting examples of biofuels obtained from the process and conveyed to deposits 230 and 240 included bioethanol, biodiesel, biomethane, biobutanol, bio-dimethyl ether, synthetic hydrocarbons, biohydrogen, vegetable oils, syngas, biogas. Some biofuels in gaseous/volatile form can also be obtained from the various processes of the second stage and conveyed by suitable hermetic tubes in storage chambers 136 and, optionally after the distiller 200 or directly to the storage of gases and volatile substances 220.

### Detailed Description of the Invention in Software

The “Industrial Digestive System” herein disclosed represents a paradigm shift in the field of—primarily, but not exclusively—waste treatment and resource recovery, achieved through the seamless integration of—primarily, but not exclusively—advanced machine learning algorithms, sophisticated data analytics, and cutting-edge software engineering. This section elucidates the intricate details of the invention, providing comprehensive insight into its components, functionalities, and operational mechanisms. System Architecture: The invention is constituted of a multi-layered software architecture, designed to facilitate robust data processing, predictive analytics, and real-time or not real-time optimization of waste and or elements and or molecules treatment processes. The architecture is broadly divided into the following components: Input Analysis Module. This module is responsible for the detailed characterization or probabilistic characterization or unknown characterization of waste inputs and or elements and or molecules, utilizing a combination of sensor data, historical databases, and real-time analytics. It identifies the composition, properties, and potential contaminants present in the waste stream, providing a foundational layer of data for subsequent processing. Machine Learning Engine: At the heart of the invention is—primarily, but not exclusively—a machine learning engine, endowed with a suite of algorithms tailored for predictive modeling, pattern recognition, and optimization. The engine leverages the data procured from the Input Analysis Module, generating predictive models that ascertain the optimal chemical processes required for the conversion of waste into valuable products. Process Optimization Module. This module acts as the executor of the invention, utilizing the insights and recommendations provided by—primarily, but not exclusively—the Machine Learning Engine to optimize the various—primarily, but not exclusively—chemical, physical, mechanical, biological processes involved in—primarily, but not exclusively—waste treatment. It dynamically adjusts processing parameters,—primarily, but not exclusively—temperature, pressure, and reagent concentrations, ensuring maximal efficiency and resource recovery. Output Customization Module. Catering to the diverse needs of various application domains, this module tailors the characteristics of the output products, ensuring compliance with specific quality standards and specifications. It works in tandem with the Process Optimization Module, fine-tuning the final stages of the—primarily, but not exclusively—waste conversion process to achieve the desired product attributes. Integration and Communication Layer. Ensuring seamless interoperability with a myriad of industrial hardware configurations, this layer facilitates data exchange and command execution between the software system and the external hardware components. It supports various communication protocols and standards, ensuring robust and reliable integration. Operational Mechanism The operational mechanism of the “Industrial Digestive System” is grounded in a cyclical process of data ingestion, analysis, prediction, and optimization. The system commences operations by ingesting data pertaining to the—primarily, but not exclusively—waste input, analyzing—primarily, but not exclusively—its composition and properties. The algorithms and the—primarily, but not exclusively—Machine Learning Engine employs this data to predict the optimal processing parameters required for the transformation of the waste into valuable products. The Process Optimization Module acts on these predictions, adjusting the operational parameters of the industrial hardware. Concurrently, the Output Customization Module ensures that the characteristics of the final products align with the predefined specifications. Throughout this process, the Integration and Communication Layer maintains seamless communication between the software system and the industrial hardware, ensuring synchronized and efficient operations in real-time or not in real-time. Algorithms and Data Analytics The—primarily, but not exclusively—Machine Learning Engine incorporates a diverse array of algorithms, including but not limited to, neural networks, decision trees, and support vector machines. These algorithms are trained on extensive datasets, encompassing—primarily, but not exclusively—historical waste treatment data, transformations reaction libraries, and product specification databases. Through continuous learning and adaptation, the engine enhances its predictive accuracy and optimization capabilities, ensuring sustained performance improvements over time. Environmental and Economic Benefits. The invention unequivocally contributes to environmental stewardship and economic viability. By maximizing resource recovery and optimizing process efficiency, it significantly reduces the volumes of—primarily, but not exclusively—waste destined for landfill disposal, mitigates emissions, and conserves natural resources. Economically, the system enhances the cost-effectiveness of—primarily, but not exclusively—waste treatment operations, unlocking new revenue streams through the production of—primarily, but not exclusively—high-value products. In culmination, the “Industrial Digestive System” embodies a confluence of advanced software engineering, algorithms, machine learning, and environmental science. It addresses the multifaceted challenges of—primarily, but not exclusively—waste treatment and resource recovery, setting a new benchmark for efficiency, sustainability, and intelligent industrial processes. Through its innovative design and unparalleled functionality, the invention stands poised to revolutionize the field of waste management, heralding a future where—primarily, but not exclusively—waste is not seen as a burden, but as a valuable resource waiting to be unlocked.

### Definitions

These definitions articulate the critical components and functions of the invention, clarifying its applicability and operational scope in processing a wide array of elements, molecules, and waste materials. Machine Learning Engine: The “Machine Learning Engine,” as applied within this invention, is a computational system specifically programmed to discern patterns and features in elements, molecules, and waste materials. It applies a combination of learning algorithms to evaluate and predict the most effective strategies for converting these substances into usable resources. This adaptive engine refines its predictive capabilities over time, contributing to the progressive enhancement of waste transformation processes. Operational Mechanism: For the purposes of this patent, “Operational Mechanism” refers to the collective series of actions and processes designed to handle and transform elements, molecules, and waste. These mechanisms encompass various stages, for example: identification, classification, alteration, and conversion of the materials in question. They operate in harmony to efficiently transition the state or composition of the waste, elements, or molecules into a resourceful output. Input Analysis Module: In the context of this invention, the “Input Analysis Module” acts as the initial assessment gateway for the elements, molecules, and waste inputs. This module systematically characterizes the materials based on their inherent properties, such as chemical composition and molecular structure, enabling the system to accurately process them in accordance with their unique characteristics. Resource Recovery Algorithm: The “Resource Recovery Algorithm” is a sequence of programmed instructions tailored to identify the most valuable recovery options for elements, molecules, and waste inputs. This algorithm prioritizes the resource potential of the materials, calculating the optimal and/or most energy-efficient and sustainable pathways for their transformation into new forms, such as reusable elements, compounds, molecules or energy in different forms of matters. Computational Intelligence Layer: Within this invention, the “Computational Intelligence Layer” stands for the decision-making core that employs advanced computational techniques, like machine reasoning and pattern recognition, to enhance the transformation of elements, molecules, and waste. This layer actively fine-tunes the processing actions, ensuring that the system responds appropriately to diverse material compositions and consistently achieves optimal conversion results. Industrial Hardware Interface: The “Industrial Hardware Interface” refers to the integrated communication framework that enables the software components to direct and regulate the physical machinery involved in processing elements, molecules, and waste. This interface is designed for flexibility and compatibility with a range of industrial equipment, facilitating the translation of computational decisions into physical actions. Waste Input Compositions: In this documentation, “Waste Input Compositions” represent the variety of elements, molecules, and waste that the system is capable of processing. This encompasses all the possible states and arrangements of matter, including but not limited to solids, liquids, gases, organics, inorganics, and composites, complete with their unique and variable characteristics that the invention is tasked to analyze and transform. “Input”, as used herein, refers to any type/s of molecules, elements or substance/s, in liquid, solid or gas state, inserted at the start of the process of the present invention, can be heterogeneous or homogeneous. “Output”, as used herein, refers to any type/s of substance/s in liquid, solid or gas state, expelled at some step of the process of the present invention.

### Overview of the Process in Software.

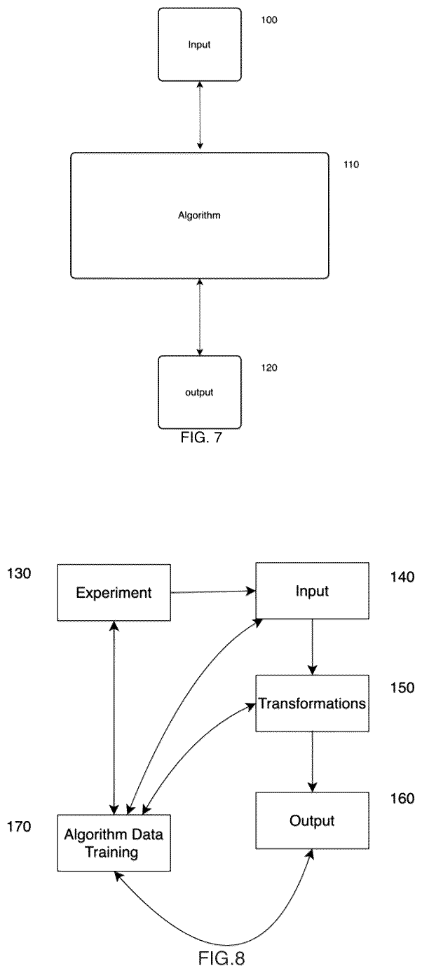

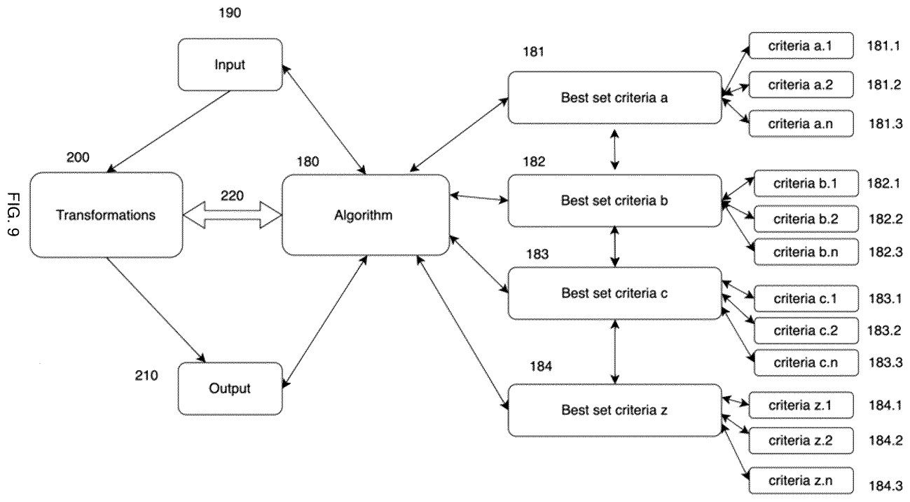

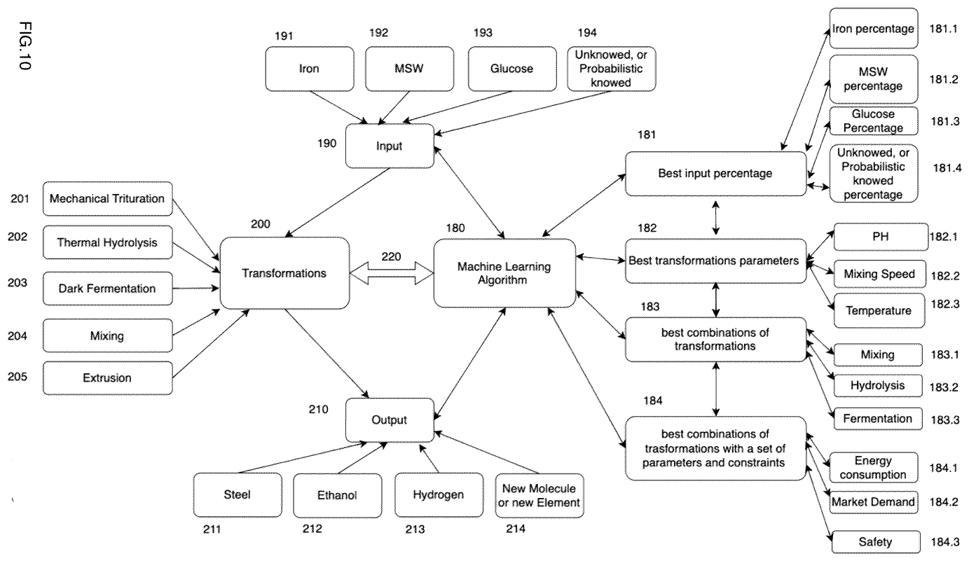

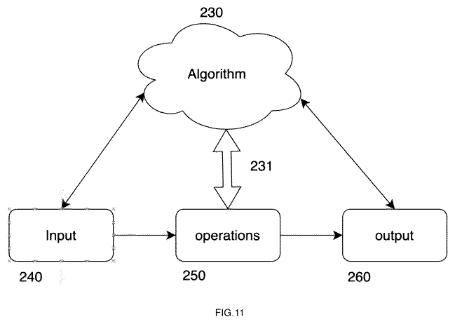

The FIG. 7 illustrates a—non limitative example-schematic representation of a processing system for the discussed invention, focusing on the flow and transformation of materials through an algorithmic function. It is a streamlined flowchart that delineates the primary stages of operation, specifically emphasizing the “Input,” “Algorithm,” and “Output” phases. Input 100: This is the initial stage of the system where raw materials are introduced. As per the invention, the input can include a variety of molecules, elements, or substances and waste in different physical states-solid, liquid, or gas. These materials can be either homogeneous, consisting of a single type of substance, or heterogeneous, containing a diverse mixture of substances of known, unknown or probabilistic known. Algorithm (110): The central part of the flowchart represents the algorithm—the core computational process of the invention. This is where the input materials are subjected to a series of operations and transformations dictated by the system's programming. The algorithm applies machine learning and other computational methods to analyze the inputs, determine the optimal pathways for conversion, and execute the necessary processes to transform the materials into valuable outputs. It is in this stage that the system's capabilities for adaptive learning, predictive analytics, and process optimization are most critical, leveraging data to refine and enhance the conversion process. Output (120): The final stage depicted in the flowchart is the output. Following processing by the algorithm, the materials emerge as transformed products. Consistent with the invention, these outputs can be new forms of matter or energy, such as renewable fuels, advanced material composites, or purified elements, available in solid, liquid, or gas states. The output represents the culmination of the invention's process, showcasing the tangible results of the sophisticated material conversion system. The flowchart provides a high-level view of the operational mechanism of the invention, capturing the essence of the input-processing-output cycle. It encapsulates the invention's fundamental process of transforming diverse materials into usable resources through the application of an intelligent algorithm. FIG. 8 presents a—non limitative example—schematic representation of the operational workflow within the “Industrial Digestive System” as delineated by the present invention. The flowchart is segregated into distinct components that collectively delineate the cyclic nature of the machine learning-driven transformation process. At the commencement of the workflow, the “Experiment” phase (130) is where empirical data is amassed. This phase is pivotal for conducting various trials with inputs under diverse conditions, allowing the system to collate data that encapsulate the transformation outcomes. Subsequent to the Experiment phase is the “Input” phase (140), where the system receives a multitude of substances. These inputs can be characterized as heterogeneous or homogeneous materials in various states-solid, liquid, or gaseous. The nature and composition of these inputs are integral to the ensuing transformation process. The “Transformations” phase (150) constitutes the core algorithmic operation wherein the input materials are subjected to an array of conversion processes. These transformations are hypothesized to encompass chemical, physical, and biological alterations, facilitating the metamorphosis of the inputs into new entities with potentially heightened utility. Upon undergoing the transformative operations, the materials reach the “Output” phase (160), which yields the end products. These outputs are envisioned as transformed states of matter or energy forms that are aligned with the objectives of resource recovery and sustainability as posited by the invention. Critical to the advancement of the system's capability is the “Algorithm Data Training” phase (170). In this phase, the machine learning engine of the system employs the data procured from both the Experiment phase and the Output phase to refine its predictive models. This iterative loop is indicative of an adaptive system that utilizes output feedback to continuously enhance its algorithms, thereby optimizing the transformation processes overtime. The flowchart encapsulates the dynamic interplay between experimental data and algorithmic processing within the “Industrial Digestive System.” It illustrates a closed-loop system where each phase informs and refines the subsequent, embodying a data-driven approach to achieving maximal efficiency in waste conversion. FIG. 9 illustrates—a non limitative example—the decision-making architecture within the “Industrial Digestive System” through an elaborate schematic of an algorithm's operational hierarchy. Each numeric label corresponds to a distinct component or process within the system: Input (190): This block represents the stage at which raw materials are introduced into the system. Consistent with the invention's definition, these inputs may include diverse substances across various states of matter. Algorithm (180): This central node is the decision-making engine, powered by machine learning. It processes the input data to determine the best approach for material transformation. Transformations (200): This stage is where the actual conversion of input materials occurs, as determined by the algorithm. The specific nature of these transformations is predicated on the input characteristics and the algorithm's decision criteria. Output (210): The transformed materials are outputted here, which may include new states of matter or energy forms suitable for utilization as resources. Feedback Loop (220): This arrow looping back from the “Transformations” to the “Algorithm” indicates that the results of the transformations feed back into the algorithm. This mechanism enables the system to learn and improve the decision-making process over time. Best set criteria a (181): This node and its subsequent branches (181.1, 181.2, 181.3) represent a decision path that the algorithm could take based on a specific set of criteria ‘a’. These criteria are likely tailored to optimize certain aspects of the transformation process. Best set criteria b (182): Similarly, this is another decision path with its own branches (182.1, 182.2, 182.3) for a different set of criteria ‘b’, illustrating the algorithm's multifaceted decision-making capability. Best set criteria c (183): This node follows the same logic, offering alternative branches (183.1, 183.2, 183.3) for criteria ‘c’, which the algorithm may select based on another set of optimization parameters. Best set criteria z (184): This represents yet another decision path, indicating the system's extensive range of criteria options (from a to z). The criteria ‘z’ and its branches (184.1, 184.2, 184.3) exemplify the comprehensive analytical scope of the algorithm. Each decision node (181-184) and its branches (0.1, 0.2, 0.3) are indicative of the system's algorithmic complexity and its ability to dissect the transformation process into multiple optimized pathways. The branches stemming from each “Best set criteria” node likely represent various levels of specificity within that decision path, refining the algorithm's instructions for the transformation processes. Overall, FIG. 3 provides a detailed—non limitative example—visualization of a complex decision-making system capable of handling a multitude of variables and outcomes, characteristic of the adaptive and intelligent nature of the “Industrial Digestive System.” FIG. 10 illustrates a comprehensive—non limitative example—flow diagram, which encapsulates the intricate processes of the “Industrial Digestive System” for converting varied inputs into valuable outputs through a series of transformation stages and algorithmic decision-making. In the upper tier of the figure, the “Input” phase (190) introduces the primary materials to be processed, including Iron (191), Municipal Solid Waste (MSW) (192), Glucose (193), and an Unknown, or Probabilistic known substance (194). These materials represent a spectrum of inputs ranging from definite, such as iron, to indeterminate, reflecting the system's versatility. The central component of the diagram is the “Machine Learning Algorithm” (180), which underpins the decision-making process. The algorithm assesses the inputs and directs the transformation processes by analyzing a multitude of variables and potential outcomes. This decision-making engine is depicted as interfacing with various nodes representing decision criteria: “Best input percentage” (181) and its sub-nodes (181.1-181.4) suggest an optimization process to determine the ideal composition of the input materials based on their respective percentages, such as iron or glucose content. “Best transformations parameters” (182) with its detailed criteria nodes (182.1-182.3), which may include parameters such as pH, mixing speed, and temperature, pivotal for optimizing the transformation processes. “Best combinations of transformations” (183) implies the algorithm's capability to identify the most effective combination of transformation processes, as detailed by the nodes (183.1-183.3), including mixing, hydrolysis, and fermentation steps. “Best combinations of transformations with a set of parameters and constraints” (184) and its sub-nodes (184.1-184.3) indicate a further level of optimization, where the system considers additional factors such as energy consumption, market demand, and safety. The “Transformations” phase (200) employs various procedures represented by nodes such as Mechanical Trituration (201), Thermal Hydrolysis (202), Dark Fermentation (203), Mixing (204), and Extrusion (205). These nodes denote specific processes that the materials undergo, each potentially contributing to the conversion into the final output. Finally, the “Output” phase (210) showcases the end products of the system's processing, including Steel (211), Ethanol (212), Hydrogen (213), and New Molecules or new Elements (214). These outputs demonstrate the diverse range of products that can be generated from the processing of various inputs. The feedback loop (220) from the “Transformations” phase back to the “Machine Learning Algorithm” suggests an iterative process, where the outcomes of transformations are used to refine the algorithm's predictive accuracy and processing efficacy continuously. In conclusion, FIG. 10 provides a detailed representation of the “Industrial Digestive System's” process workflow, illustrating the complex interplay between inputs, algorithm-driven decision-making, transformation processes, and outputs. The system's design embodies a sophisticated approach to resource recovery, emphasizing adaptability, optimization, and the integration of complex data analytics to enhance the conversion of materials into a broad array of valuable products. FIG. 11 portrays a flowchart conceptualizing the integration of an algorithm with hardware components within the “Industrial Digestive System” as per the present invention. The flowchart is segregated into distinct elements, each symbolizing a pivotal phase in the hardware-software interfacing process. At the outset, the “Input” block (240) is depicted as the initial interface where input and related data are fed into the system. This block is crucial for initiating the conversion process, representing the entry point for materials to be transformed. Central to the diagram is the “Algorithm” cloud (230), illustrating the abstract nature of the computational process. This symbolizes the machine learning algorithm's role as the decision-making entity that dictates the subsequent operations on the input materials and related data. The cloud representation underscores the algorithm's non-tangible, computational aspect, distinguishing it from the physicality of the inputs and outputs. Proceeding from the algorithm is a bidirectional arrow (231) leading to and from the “Operations” block (250), indicating a dynamic interchange between the computational decisions and the physical transformation processes. This exchange reflects the system's ability to translate algorithmic decisions into physical actions as well as to relay the results of these actions back to the algorithm for further analysis and refinement. The “Operations” block (250) is where the physical processing or transformation of materials occurs, as governed by the algorithm. This block embodies the various mechanical, chemical, or biological processes that the input undergoes to convert it into the desired output. Culminating the process is the “Output” block (260), which signifies the end product of the system's operations. The outputs are the tangible results of the input materials' transformations, exemplifying the system's ultimate purpose of converting inputs into valuable resources or products. In summation, FIG. 11 encapsulates the integrated workflow of the “Industrial Digestive System,” highlighting the interplay between the software-driven algorithm and the hardware-implemented operations that together enable the conversion of inputs into outputs. The flowchart underscores the system's design, which embodies a sophisticated feedback loop between hardware and software, ensuring that the physical operations are continually optimized based on the algorithm's computations. In the context of the “Industrial Digestive System” as described by the present invention, an exemplary mathematical model is posited to serve as the foundation of the algorithm which governs the transformation processes. The model integrates sets, criteria, weights, and matrix representations to calculate the optimal conversion pathways for input materials into specified outputs. The set \(P=\{p_1, p_2, . . . , p_n\}\) represents the ensemble of transformation processes available within the system, encompassing \(n\) distinct processes. \(M=\{m_1, m_2, . . . , m_I\}\) denotes the set of input materials, with \(I\) individual materials subjected to the transformation processes. For each potential combination \(k\) of transformation processes, \(C_k=\{ck_1, ck_2, . . . , ckm\}\) encapsulates the selected criteria that are evaluated to optimize the transformation process. Correspondingly, \(C_{i_k}=\{c1_k, c2_k, . . . , cm_k\}\) represents the set of criteria for each process \(p_i\) in combination \(k\), where the criteria are assessed to ensure the efficacy of the transformation. The model employs a set of criteria weights (W=\{w_1, w_2, . . . , w_m\}\), with each weight \(w_i\) corresponding to the importance or influence of criteria \(c_i\) in the decision-making process. The optimal outputs are defined as \(O_n=, fixed objectives that the transformation process aims to achieve. The optimal percentage of input materials is represented by \(X=\{x_1, x_2, . . . , x_I\}\), which denotes the proportion of each material \(m_i\) that contributes to the optimal functioning of process \(p_i\) in combination \(k\). The matrix \(A=[a_{ij}]\) quantifies the amount of output product \(o_j\) generated from utilizing one unit of material \(m_i\) in process \(p_i\). The model further includes \(V=\{v_1, v_2, . . . , v_r\}\), delineating the best variables for each chemical combination within the transformation process. The score function \(S(C_k, X)=\Sigma (\Sigma (w_i*c_{i_k}(p_i)))+\Sigma (\Sigma (x_i*a_{ij}*o_j))\) is a composite function that assesses the suitability of combination \(k\) and material percentages \(X\) by considering both the criteria weights and the output products.

$$S ( {C}_{m} , {X}_{m} ) = {\sum }_{i}^{n} [ {\sum }_{j}^{n} ( {W}_{i} * {C}_{{i}_{k}} ( {P}_{i} ) ) ] + {\sum }_{i}^{n} [ {\sum }_{j}^{n} ( {x}_{i} * [ \begin{matrix}{a}_{11} & … & {a}_{1 j} \\ ⋮ & ⋱ & ⋮ \\ {a}_{i 1} & … & {a}_{ij}\end{matrix} ] * {o}_{j} ) ]$$

The optimal configuration \((C_{optimal}, R_{optimal}, V_{optimal}, P_{optimal})\) is obtained by minimizing the score function across all possible combinations, where the aim is to find the set of transformation processes, criteria combinations, variables, and input material percentages that yield the minimum discrepancy between the properties of the output products and the predefined optimal outputs.

$$Optimal = ( {C}_{best} {R}_{best} {V}_{best} {P}_{best} ) = arg min \{ S ( {C}_{1} {X}_{1} ) , S ( {C}_{1} {X}_{1} ) , … , S ( {C}_{m} {X}_{m} ) \}$$

This mathematical model reflects a multifaceted approach to optimizing the conversion of input materials into valuable resources, leveraging a combination of weighted criteria and matrix-based output estimations to facilitate decision-making within the “Industrial Digestive System.” The mathematical model under discussion, \(\varepsilon_{mi}\hat{ }{Pn}\), is posited as the mathematical underpinning of the algorithm within the “Industrial Digestive System”. The model is designed to capture the essence of the transformation processes applied to various materials and their respective properties. The transformation index \(P_n\) is formulated as the sum of the products of \(alpha_k\) and \(V_j\), where \(\alpha_k\) represents a coefficient corresponding to the \(k\hat{ }{th}\) transformation process, and \(V_j\) denotes a variable associated with the (j\hat{ }{th}\) transformation process. The index \(m\) indicates the specific material under consideration, and \(i\) represents a property of the material that is subject to transformation. The final state of a material or system, \(D_{final}\), is then expressed as the product of the transformation effects \(\varepsilon_{mi}\hat{ }{Pn}\) for each process \(P_n\), applied sequentially to the initial state \(D_{initial}\). This recursive product implies that the effect of each transformation process on the material's property is cumulative and multiplicative. The expression \(D_{final}=\varepsilon_{mi}\hat{ }{p1}\cdot \varepsilon_{mi}\hat{ }{p2}\cdots \varepsilon_{mi}\hat{ }{pn}\cdot D_{initial}\) further expands this concept, illustrating that the final state is the result of applying each individual transformation effect \(\varepsilon_{mi}\hat{ }{Pn}\) to the initial state, in a sequence from the first process \(\varepsilon_{mi}\hat{ }{p1}\) to the \(n\hat{ }{th}\) process \(\varepsilon_{mi}\hat{ }{pn}\). The objective function, \(\text{min}\sum (D_{final\_predicted}−D_{final\_observed})\hat{ }2\), is a classic minimization problem where the goal is to minimize the sum of squared differences between the predicted final state and the observed final state.

$$min \sum ( {D}_{\{ {final}_{predicted} \}} - {D}_{\{ {final}_{observed} \}} ) \hat{} 2$$

This represents an optimization routine where the algorithm iteratively adjusts the transformation processes to minimize the discrepancy between the predicted and observed outcomes. In essence, this model provides a structured approach to quantitatively assess and optimize the transformation processes by predicting the material properties post-transformation and refining the processes to align with observed data. It embodies the integration of computational algorithms with empirical observations, central to the adaptability and efficiency of the “Industrial Digestive System”. The mathematical element \(\varepsilon_{mi}\hat{ }{P_n}\), referred to as the action tensor or transformation tensor within the context of the “Industrial Digestive System,” encapsulates the multi-dimensional effects of the transformation processes on various material properties from the initial state \(D_{initial}\) to the final state \(D_{final}\).
eₘᵢ^{P}^{ₙ}

This tensor can be viewed as a high-dimensional array where each element is associated with a specific transformation process \(P_n\) applied to a particular material \(m\) and affecting a certain property \(i\). The indices \(m\), \(i\), and \(P_n\) represent the dimensions in the material space, property space, and process space, respectively. In the system described by the present invention, the action tensor serves the following roles: 1. Multi-dimensional Mapping: It maps the effects of each transformation process across various materials and their properties, creating a comprehensive model of potential outcomes. 2. Cumulative Effect Representation: The tensor's structure allows for the representation of cumulative effects, where the impact of sequential transformations on a material's property is accumulated across the process chain. 3. Process Interaction: The tensor accounts for interactions between different processes, where the effect of one transformation may be dependent on the preceding transformations. The action tensor is fundamental in computing the final state \(D_{final}\) of the material after undergoing a sequence of transformations. It provides a mathematical framework that integrates empirical data and machine learning predictions to optimize the process parameters, ensuring the conversion from \(D_{initial}\) to \(D_{final}\) is as efficient and effective as possible. In practice, the action tensor would be populated with values derived from both theoretical modeling and experimental data. These values characterize how each transformation (e.g., heating, mixing, chemical reaction) alters a material property (e.g., viscosity, concentration, phase). The tensor thus embodies the transformational knowledge required by the algorithm to simulate and predict the outcomes of the industrial processes described in the invention.

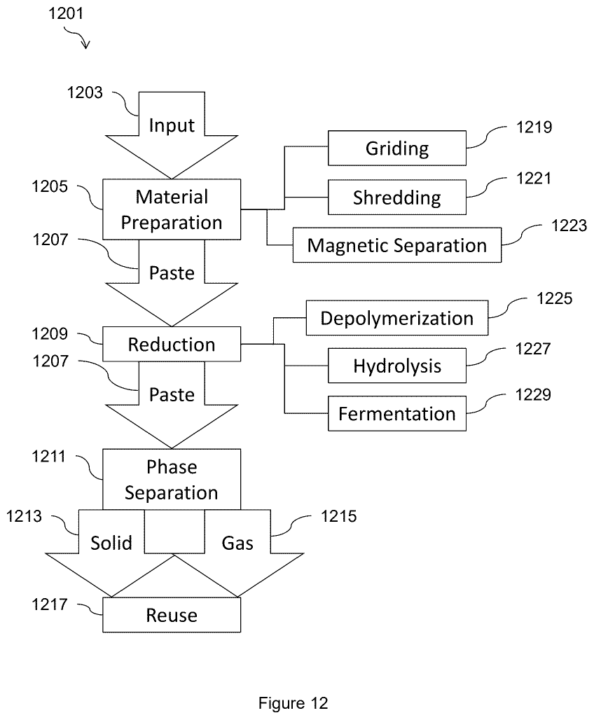

Referring now to FIG. 12 an embodiment 1201 of the industrial digestive system includes multiple processing stages, for example in the current embodiment they are material preparation 1205, reduction 1209, and phase separation 1211. Input waste 1203 enters material preparation 1205 which has the potential to subject input waste 1203 to grinding 1219, shredding 1221, and magnetic separation 1223. Input waste 1203 exits material preparation 1205 as paste 1207. The viscosity and material properties are altered by the system of embodiment 1201. Paste 1207 proceeds as directed to reduction 1209 which has the potential to subject paste 1207 to depolymerization 1225, hydrolysis 1227, and fermentation 1229. It is contemplated that when paste 1207 exits reduction 1209 that it is substantially ready to be isolated into usable materials via phase separation 1211. The solids 1213 and gases 1215 of paste 1207 are separated and rendered in their preferred forms for reuse 1217. The system of embodiment 1201 has the capability to select and subsequently direct input waste 1203 to any or none of the processing stages as determined by algorithmic optimization. It is contemplated that the stages are interconnected as are the processes which make up each of the stages. It will be appreciated that this interconnectedness is what enables embodiment 1201 to receive input waste 1203 materials in the form of homogeneous, heterogeneous, molecular, elemental, miscellaneous, or uncategorized waste, and classify them based on their physical and chemical properties to render them usable again.

It is further contemplated that a control system is implemented within embodiment 1201 to facilitate its algorithmic optimization. The control system is configured to monitor the input waste 1203, the operation of each of the stages and subprocesses, the respective output from each of the stages and subprocesses. The data thusly obtained is used to populate the algorithms used to optimize the flow, sequence, and output of embodiment 1201.

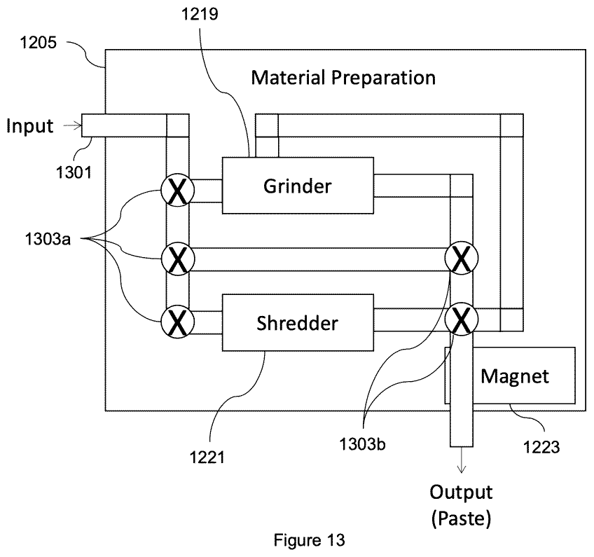

Referring now to FIG. 13, an exemplary embodiment of a material preparation stage is illustrated. In the depicted embodiment, the material preparation stage 1205 is configured to receive input waste 1301. The material preparation stage 1205 is structured to selectively subject input waste 1301 to a grinding unit 1219 and/or a shredding unit 1221 arranged to reduce particle size and facilitate downstream processing and/or directly to a magnet 1223 that extracts magnetic material from input waste 1301.

The material preparation stage 1205 further comprises a series of controllable conduits and flow diverters represented symbolically, for example at 203a and 203b, these enable the selective routing of material to and from grinder 1219 and shredder 1221, as well as bypassing either or both units if desired.

Additionally, the material preparation stage 1205 is operatively coupled to a magnetic separation device 1223. The magnetic separation device 1223 is configured to remove ferromagnetic and paramagnetic contaminants from the processed material. As such, upon completion of grinding, shredding, and/or magnetic separation, the material exits material preparation stage 1205 in the form of a homogenized and substantially contaminant-reduced paste. This paste may then be directed onward for subsequent processes such as reduction, depolymerization, hydrolysis, fermentation, or other downstream treatments as described in preceding and subsequent figures.

In this embodiment, the presence of multiple processing units (e.g., grinder 1219, shredder 1221, and magnet 1223) and the associated controllable conduits (e.g., 203b, 203a) facilitate an algorithmically optimized configuration whereby the selection of units and material flow paths can be dynamically adjusted. These adjustments are based on monitored material properties and performance metrics, enabling the system to tailor the processing of heterogeneous, homogeneous, and otherwise uncategorized input waste for optimal output quality, system efficiency, and material recovery.

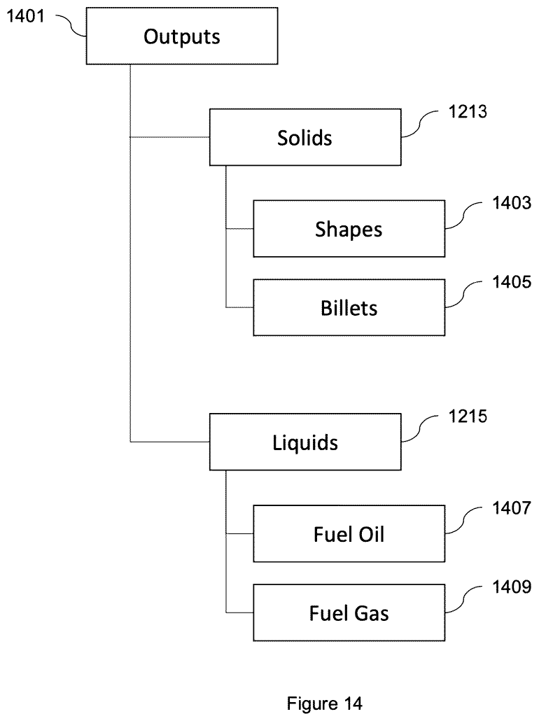

Referring now to FIG. 14, an exemplary embodiment of the output streams generated by the industrial digestive system is illustrated. In the depicted embodiment, the outputs 1401 are categorized into primary material classifications, such as solids 1213 and liquids 1215, which represent distinct end-products derived from potentially heterogeneous, molecular, or elemental input waste streams.

In one aspect, the solids 1213 may be further processed into standardized or customized forms. For example, the solids 1213 can be molded, pressed, or otherwise formed into shapes 1403 or billets 1405. These shaped solid outputs constitute composite materials, conglomerates, or other reusable structural forms that can be deployed as feedstock for various industrial applications, repurposed into consumer products, or integrated into supply chains requiring intermediate solid materials.

In another aspect, the liquids 1215 produced by the industrial digestive system include fractions that may be refined into valuable energy carriers or chemical precursors. In the illustrated embodiment, the liquids 1215 are shown partitioned into fuel oil 1407 and fuel gas 1409. Fuel oil 1407 serve as a combustible fuel resources or as a feedstock for downstream chemical processing, whereas fuel gas 1409 and fuel oil 1407 may include diesel fuel, heating oil, bunker fuel, kerosene, biofuel, natural gas, propane, butane, biogas, synthetic gas, hydrogen, ammonia, methanol, liquid organic hydrogen carriers, dimethyl ether, furnace oil, gasoil, jet fuel, residual oil, LPG (Liquefied Petroleum Gas), town gas, ethane, hydrogen sulfide, aviation gasoline, naphtha, gas condensates, wood gas, acetylene, hydrogen chloride, LNG (Liquefied Natural Gas), CNG (Compressed Natural Gas), peat, coke, blast furnace gas, refinery gas, oil shale, tar sands oil, ethanol, biodiesel, algal biofuel, synthetic diesel, coal gas, producer gas, lignite, bitumen, vegetable oil, methanol fuel, butanol, synthetic natural gas (SNG), wood pellets, torrefied biomass, petroleum coke, lignite coal, sub-bituminous coal, torrefied biomass, cellulosic ethanol, renewable diesel, renewable natural gas, compressed biogas, synthetic natural gas, propane autogas,
or other gaseous or liquid fuel products suitable for combustion, power generation, or further refinement into specialty chemicals.

The depicted embodiment highlights the versatility of the system in both managing diverse input wastes and delivering multiple, usable outputs, including composites with thermoplastic properties, biofuels, biogas, chemical substances, and other valuable resources. The system's control and optimization capabilities, informed by monitoring the input characteristics, operational parameters, and output qualities, allow for dynamic adjustments that maximize yield, enhance efficiency, and broaden the scope of potential industrial applications for the recovered materials.

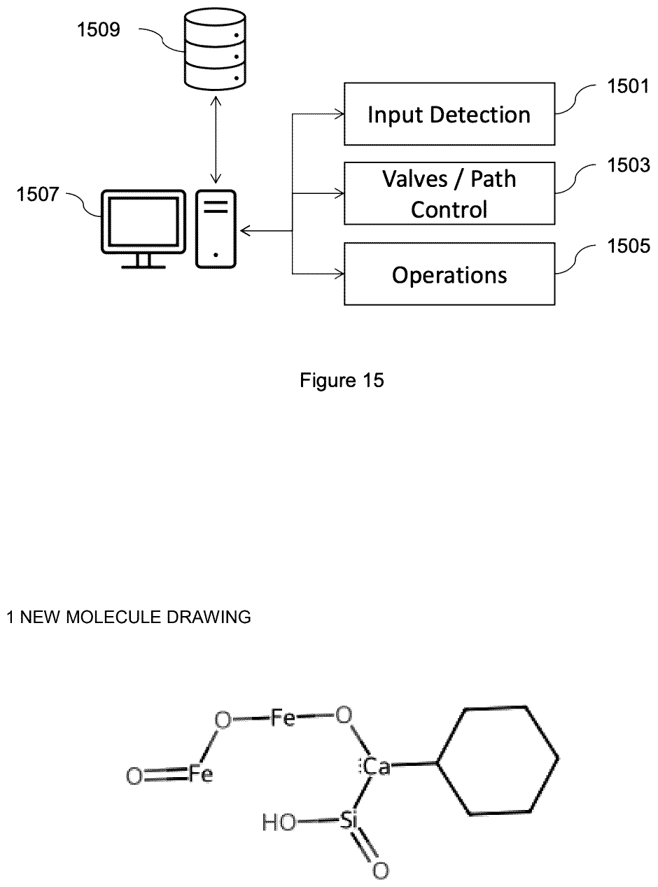

Referring now to FIG. 15, an exemplary embodiment of a control system architecture for an industrial system is depicted. In accordance with the claims, the control system operates within a digital environment and leverages both computational and sensing elements to optimize the processing and reclamation of input waste.

In the illustrated exemplary embodiment, the control system includes an input detection module 1501, which is configured to receive signals from at least one sensor and identify characteristics of the incoming input materials. This sensor-driven identification serves as the foundational input for the optimization module, enabling the system to classify the input waste as homogeneous, heterogeneous, molecular, elemental, miscellaneous, or uncategorized.

The control system further comprises a valves/path control module 1503. This module is associated with at least one actuator that governs the flow of material through various subprocesses within the waste reclamation system. Based on the sensed input properties and instructions generated by the optimization module, the valves/path control 1503 dynamically directs input waste along selected routes. For example, it can open or close valves, engage routing diverters, or alter the operational sequence within the reclamation stages to ensure optimal resource extraction and waste conversion efficiency.

Also shown is an operations module 1505, which represents the functional implementation of processes performed on the input materials. These processes may include grinding, shredding, depolymerization, hydrolysis, fermentation, and phase separation. The operations module 1505 executes instructions relayed from the optimization module, adjusting its parameters in real-time to enhance system performance and resource yield.

The entire control framework is integrated with a computing infrastructure (represented by the computer terminal 1507) and a database 1509. The database 1509 provides digitally accessible criteria, reference data, historical operational metrics, and learned patterns from previous waste processing runs. The optimization module, which resides in the control system and interfaces with the CPU and memory resources, uses this stored data in conjunction with machine learning and/or quantum algorithms. This adaptive learning system updates its predictive models and optimization strategies over time, improving the accuracy of waste identification, routing decisions, and operational parameter selections.

Through a feedback loop, performance metrics and outcomes from the operations module 1505 are continually fed back into the database 1509 and analyzed by the optimization module. This iterative process refines the decision-making models, ensuring that the system steadily improves its efficiency and resource recovery capabilities. In other words, the observed results guide subsequent adjustments, allowing the system to handle diverse input wastes and multiple output demands, as specified by the claims.

In essence, FIG. 15 illustrates the orchestrated interplay between sensing (input detection 1501), actuation (valves/path control 1503), process execution (operations 1505), computational intelligence (the central computing and machine learning environment 1507), and data storage/retrieval (database 1509). This synergy underpins the control system's adaptive optimization of the waste reclamation process, producing valuable outputs such as biogas, biofuels, chemicals, and composite materials.

### Non-Limiting Example

Non-imitative Example of Software-Hardware Integrated Process. Creation of bioethanol, chemicals, and a new molecule and related composite materials (including the CHELONIA MBH), from molecules, elements and potentially considerably uncategorized & miscellaneous biomass & waste, integrating software and hardware of the industrial digestive system. INPUT: molecules, elements and potentially considerably uncategorized & miscellaneous biomass & waste.

TAB INPUT

| | | Primary | Elements/ | Potential Liquid |
|---|---|---|---|---|
| Material_ID | % | Materials | Compounds | Materials |
| 1, M_msw | 0.25 | Organic materials, | Carbon, Hydrogen, | Organic liquid |
| | | plastics, paper, | Oxygen, Nitrogen | waste, liquid |
| | | metals, glass | | from discarded |
| | | | | food and |
| | | | | beverage |
| 2, M_plastic | 0.4 | Various types of | Carbon, Hydrogen, | Liquid resins |
| | | plastic | Bromine, Chlorine | used in plastic |
| | | | | manufacturing |
| 3, M_construction | 0.1 | Concrete, wood, | Calcium, Silicon, | Water, oils and |
| | | drywall, asphalt, | Aluminum, Iron | lubricants used |
| | | metals, plastic | | in machinery |
| 4, M_silica | 0.025 | Glass, with different | Silicon Dioxide, | Water, liquid |
| | | additives depending | Sodium Carbonate, | glass during |
| | | on type | Calcium Oxide, | manufacturing |
| | | | Aluminum Oxide | |
| 5, M_textile | 0.02 | Natural fibers (like | Carbon, Hydrogen, | Dyes and other |
| | | cotton and wool), | Oxygen, Nitrogen | liquid chemicals |
| | | synthetic fibers (like | | used in textile |
| | | polyester and nylon) | | production |
| 6, M_agricultural | 0.15 | Plant residues, | Carbon, Hydrogen, | Animal waste |
| | | animal waste | Oxygen, Nitrogen, | liquids, |
| | | | Phosphorus, | pesticide runoff |
| | | | Potassium, Glucose | |
| 7, M_Iron | 0.05 | Various metals, | Iron | Coolants, |
| | | plastics, circuit | | lubricants |
| | | boards | | |
| 8, M_hazardous | 0.005 | Heavy metals, | Lead, Mercury, | Solvents, oils, |
| | | solvents, acids, | Cadmium | certain |
| | | alkalis, radioactive | | pharmaceuticals |
| | | substances | | |

Software Input Specification (FIG. 7, 100) and equivalent in (FIG. 1, n.1 stage, 100). The process commences with a precise quantification of input materials, where each type, denoted by Material_ID, is meticulously analyzed in correspondence of FIG. 7, 120 optimum required output. The system's inputs range across various substance categories—organic, plastic, construction elements, and more as a molecules, compounds, elements or miscellaneous and uncategorized biomass and waste—each with a calculated percentage allocation provided by the system's optimization algorithm in relation of FIG. 7, 120 optimum required output. This targeted percentage is crucial for achieving the desired output, in this non limitative example set as steel-like material and bioethanol. Algorithmic Optimization (FIG. 7, 110) or equivalent the mathematical element \(\varepsilon_{mi}\hat{ }{P_n}\), referred to as the action tensor or transformation tensor or equivalent the algorithm cloud in FIG. 11 230, is at the core of the system lies the algorithmic function, which, upon receiving the inputs, provide the respective optimal percentages and determines the most efficient conversion pathways. It is engineered to adaptively learn and predict, optimizing the process flow to transform the identified substances into the predetermined optimum outputs. The algorithm intricately balances the input percentages to align with the goal of producing, in this non limitative example steel-like and bioethanol outputs. Output Realization (FIG. 7, 120): Following the algorithm's processing (as in algorithm cloud in FIG. 11 230), the inputs FIG. 11 240, are transmuted into the anticipated outputs FIG. 11 260, with hardware operations FIG. 11 250, connected with the cloud thanks to FIG. 11 230. The precise percentages of materials, transformations, combinations of transformations and combinations of transformations with a set of criteria and parameters provided earlier in (FIG. 7 110): culminate in the production of in this non limitative example: steel-like material and bioethanol, demonstrating the algorithm's capacity to predict and suggest the transformation or operations FIG. 11 250 of diverse inputs into specifically targeted valuable output. In particular, in relation to the FIG. 9, the Industrial digestive system (FIG. 1, n.1 stage, 100) considers:
the Input \(M=\{m_1, m_2, . . . , m_I\}\) (FIG. 9, 190): {1, M_msw; M_plastic; 3, M_construction; 4, M_silica; 5, M_textile; 6, M_agricultural; 7, M_Iron; 8, M_hazardous}
the output O_n (FIG. 9, 190), fixed objectives that the transformation process aims to achieve—In this non limitative example steel-like and bioethanol —, through the optimization algorithm FIG. 9, 180). therefore the optimization algorithm (FIG. 9, 180), elaborate the
optimal input percentages \(X=\{x_1, x_2, . . . , x_I\}\), (FIG. 9, 181), in this non limitative example as showed in Tab percentage:

Tab percentage.

| Material_ID | % |
|---|---|
| 1, M_msw | 0.25 |
| 2, M_plastic | 0.4 |
| 3, M_construction | 0.1 |
| 4, M_silica | 0.025 |
| 5, M_textile | 0.02 |
| 6, M_agricultural | 0.15 |
| 7, M_Iron | 0.05 |
| 8, M_hazardous | 0.005 |

the best transformations parameters \(P=\{p_1, p_2, . . . , p_n\}\) and variables \(V=\{v_1, v_2, . . . , v_r\}\), (FIG. 9, 182), in this non limitative example as showed in tab transformations:

tab transformations

| Process_ID, | Waste | Temperature | Pressure | Time |
|---|---|---|---|---|
| Process_Name | Input | (° C.) | (atm) | (min) |
| 1, V_trituration | All waste | 25 | 1 | 30 |
| 2, V_Hidrolysis (Thermal) | MSW + biomass | 120 | 2.5 | 30 |
| 3, V_fermentation | MSW + biomass | 25 | 1 | 50000 |
| 4, V_Separation | MSW + biomass | 25 | 1 | 40 |
| 5, V_heating | Solid fraction | 120 | 3 | 37 |
| 6, V_mixing | Solid fraction | 80 | 5 | 37 |
| 7, V_extrusion | Solid fraction | 100 | 7 | 30 |
| 8, V_distillation | Liquid fraction | 100 | 1 | 60 |

the best combinations of transformations \(C_k=\{ck_1, ck_2, . . . ckm\}\) (FIG. 9, 183), in this non limitative example as showed in tab combinations:

Tab combinations
Combination
Process_ID, Process_Name

#1.
1, 2, 3, 4, 5, 6, 7, 8

the best combinations of transformations with a set of criteria and parameters [\(C_{i_k}=\{c1_k, c2_k, . . . , cm_k\}\) represents the set of criteria for each process \(p_i\) in combination \(k\),](FIG. 9, 184), in this non limitative example as: minimization of the energy consumption and maximization of bioethanol production. Finally the optimization algorithm (FIG. 9, 180) or equivalent the Algorithmic Optimization (FIG. 7,110) finalize the best criteria [a,n; b,n; c,n; . . . ; z,n] or equivalent the optimal configuration \((C_{optimal}, R_{optimal}, V_{optimal}, P_{optimal}) and then trough 220 integration, suggest to the hardware, the best workflow or equivalent, the minimization of: the score function \(S(C_k, X)=\Sigma (\Sigma (w_i*c_{i_k}(p_i)))+\Sigma (\Sigma (x_i*a_{ij}*o_j))\) or the \(\text{min}\sum (D_{final\_predicted}−D_{final\_observed})\hat{ }2\), sum of squared differences between the predicted final state and the observed final state. An average skilled in the art is able, with modern instrumentation, to understand how the inputs and the optimal outputs are classified, that is, of course, in relation to their mechanical, thermal, chemical and physical characteristics and properties. After analyzing the inputs, we proceed with the hardware, integrating the software with the logical process of the industrial digestive system, following the combinations, variables, percentages, and other criteria provided by the software of the industrial digestive system. —In this non limitative example—we triturate the inputs described in the Input table to increase their surface volume, thus facilitating subsequent transformations (FIG. 1, stage 2). This non-limitative procedure is schematically represented in FIG. 1, n.110, 120, foreseeing the triturating of the inputs following different criteria, such as the type of trituration (destruction of inputs in a range from 0.1 to 1 cm), as well as the type of mechanical trituration (filamentary, spherical, etc.). Secondly, we proceed with hydrolysis, a process described in stage.2, hardware 130.b, which allows the inputs to reduce the bacterial load and to promote the efficacy of subsequent processes, such as fermentation. For example, in a non-limitative experiment, thermal hydrolysis was conducted with a pressure between 1 MPa and 10 MPa, a temperature between 60 Celsius and 250 Celsius, and a time between 10 minutes and 800 minutes. The subsequent process, fermentation, described in stage.2, hardware 130.b, facilitates the conversion of biomass into biofuels and volatile substances. For example, the non-limitative experiment included for anaerobic fermentation a pressure between 1 MPa and 10 MPa, a temperature between 10 Celsius and 200 Celsius, and a time between 10 hours and 1 month, in addition to the use of a mix of bacteria, including Saccharomyces cerevisiae, with a pH between 4 and 7. Subsequently, as described in stage 3, hardware, n.140, we separate the paste into liquid and solid components. The separation between liquids and solids can occur in different ways, including, but not limited to, the use of filters with various sizes between 100 microns and 1000 microns, an applied pressure between 1000 Pa and 10 Mpa, and a temperature between 10 Celsius and 45 Celsius, using centrifuges of various natures. Subsequently, the solid part (FIG. 1, 145), integrated with some inputs from the input table, is subjected to FIG. 1, stage 4, n.140, 150, which includes heating and mixing the solid paste. This transformation gives rise to a composite material with improved mechanical, thermal, and chemical properties, measurable through classical instrumentation. This transformation included, for example, a mixing speed variable between 20 and 3000 RPM, a rate of thermal input between 2 and 20 Celsius per minute, a mixing temperature between 40 and 340 Celsius, and a mixing time between 20 minutes and 4.5 hours. The result of this transformation is then subjected to the step FIG. 1, 170 of extrusion, which, in this non-limitative example, exploited the use of mechanical pressure extruders, with values between 4 Pa and 5000 Pa. The liquid and/or gaseous fraction (FIG. 1, 180) was then treated with distillation (FIG. 1, stage 5, n. 200), which in this non-limitative example was conducted using a double reactor distiller with a constant exchange of liquid, at room temperature (10-30 Celsius). The distillation was performed with a feed rate between 0.2 and 3 liters, an operating pressure of 1 atm, temperature profiles between 60 and 120 Celsius, and concentration profiles in number of moles, determinable by an expert in the field, based on the input table. Thanks to the procedures illustrated in this invention, and thanks to the proposed non-limitative experiment, we have obtained the following output products: a composite material product (FIG. 1, n. 180) in three different forms, including one potentially suitable for the construction industry, the capital letter C of Chelonia, and a tubular profile, demonstrating the versatility of the products obtained. Additionally, 22 ml of bioethanol (FIG. 1, 230) with a concentration of pure ethanol at 19%, and various chemical compounds (FIG. 1, 240, 250) were produced. The cubic composite material, named CHELONIA MBH produced through the non-limitative experiment shows remarkable properties. Mainly low density and ultra-light characteristics. Notable magnetic properties, demonstrating that the bioplastic matrix has been reinforced and bound to other components, including iron. Waterproof properties. Medium flexibility and low electrical conductivity.
   Non-Limitative Example of Advanced Composite Material Produced with the Subject of the Present Invention: Chelonia MBH Description

Categories: Polymer, Biopolymer, Thermoplastic, Carbon filler filled, Metal filler filled, Glass filler filled, Hazardous material filled. Material notes: The advanced composite material synthesized through the INDUSTRIAL DIGESTIVE SYSTEM methodology constitutes a thermoplastic matrix integrating heterogeneous biopolymers with high-performance constituents, including glass, metallic, and textile fibers. Predominantly, the composite is formulated with polyethylene (PE) and an assortment of non-classified organic compounds. This biopolymeric matrix imparts exceptional plastic behavior under compressive forces, attributable to its low-density configuration. Further enhancement of these properties is facilitated by the incorporation of carbon fibers and glass-metal-concrete fillers. Consequently, the material exhibits great potential for utilization in various sectors, including seismic-resistant construction, automotive, and aerospace industries, offering a unique combination of strength, resilience, and ultralightweight characteristics. Moreover, the presence of metallic constituents within the composite enables its application in radiation-shielding aerospace components, providing protection against diverse radiation types. This advanced material, thus, demonstrates a versatile and innovative approach to addressing multifaceted engineering challenges in an array of industries. Product Benefits: Exceptional elastic behavior under compressive forces. Low-density configuration, resulting in an ultralightweight material. High strength and resilience, suitable for seismic-resistant construction. Broad applicability in automotive and aerospace industries. Radiation shielding capabilities due to the presence of metallic constituents. Low electrical conductivity, making it suitable for electrical insulation applications. Waterproof, providing resistance to water intrusion and enhancing durability. Magnetic properties, enabling potential use in electromechanical devices and sensors. Low flammability, contributing to improved fire safety in various applications. Low heat conductivity, providing thermal insulation and energy efficiency. Resistance to chemicals, enhancing material longevity and resilience against corrosive environments. Process Benefits: Waste valorization. Versatile feedstock. Classical extrusion process. Recyclability and reusability. Energy efficiency; Reduced environmental impact. In particular, the documentation of a quantitative analysis carried out in the laboratory is attached. It is also attached, the basic molecule result, related to the new composite material.

| Category | Elements | Percentage |
|---|---|---|
| Matrix | CHO | 40-60% |
| Particle Reinforced | CaFeSiCHO | 60-40% |
| Empirical Formula | Fe₈Si₅O₂₂C₂₈₂H₅₃₄Ca₅ | |

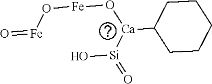

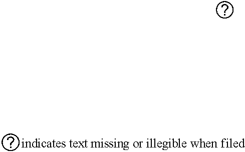

Algorithm Training Through Experimentation (FIG. 8). In the context of the present invention, the experimentations phase serves as a crucial foundation for training the optimization algorithm, as illustrated in (FIG. 8).

### Algorithm Optimization Through Empirical Data

The present invention harnesses empirical data derived from controlled experiments to train the machine learning algorithm, as the non-limitative example provided, thereby enhancing the optimization of the input transformation process. FIG. 8 demonstrates the intricate relationship between the experimental phase and the subsequent algorithm training. Experimentation (FIG. 8, 130): The experimentation phase is an integral component of the invention's methodology. It involves the systematic collection and analysis of data from the processing of a diverse range of input, as identified in the input stage. This stage is instrumental in establishing baseline parameters and transformational outcomes for various input types, such as municipal solid waste (MSW), plastics, and other. Input Analysis (FIG. 8 140): following the initial data collection, the inputs are introduced to the system, encompassing a broad spectrum from organic matter to hazardous substances. Each material type is meticulously characterized to inform the transformation process. Transformation Process (FIG. 8, 150): data from these transformations provide a rich dataset for the algorithm to learn from, ensuring that the system can accurately predict and optimize the processing parameters for each type of input material. Algorithm Data Training (FIG. 8 170): Crucially, the collected data from the experimentation and transformation phases feed into the algorithm data training component. Here, the optimization algorithm is refined using the outcomes and conditions of the transformations. This iterative training process enables the algorithm to improve its predictive accuracy and efficiency, adapting to the complexities of the input materials and desired output specifications. Output Generation (FIG. 8, 160): the output phase is the culmination of the process, where the trained algorithm's efficiency is realized through the production of target output. The quality and characteristics of these outputs validate and set the effectiveness of the algorithm's training. In conclusion, the experimentally informed training of the optimization algorithm is pivotal to the invention's ability to transform inputs into valuable outputs with precision. This systematic approach to algorithm training ensures continuous improvement of the system, thereby enhancing the overall efficacy of the “Industrial Digestive System.”

What is claimed is:

1. An industrial digestive system, comprising: a plurality of interconnected processing stages, each stage configured to perform specific transformations on input waste materials to alter their physical, chemical, biological or state properties; an intake and automated sorting apparatus configured to receive input waste materials in the form of homogeneous, heterogeneous, molecular, elemental, miscellaneous, or uncategorized waste, and classify them based on their physical and chemical properties; a control system that monitors the input, function, and output of the system and each of the plurality of stages, directing the input waste through optimized processing pathways to reclaim usable outputs; wherein the input waste is sorted and then processed as optimized by the control system to reclaim usable outputs.

2. An advanced composite material synthesized through an industrial digestive system methodology replicating biological digestive processes, comprising: a thermoplastic matrix integrating heterogeneous biopolymers, predominantly formulated with polyethylene and an assortment of non-classified organic compounds, wherein the matrix comprises 40-60% by weight of elements carbon (C), hydrogen (H), and oxygen (O); high-performance constituents dispersed within the matrix, including glass fibers, metallic fibers, textile fibers, carbon fibers, glass-metal-concrete fillers, and hazardous material fillers, wherein the particle reinforcements comprise 60-40% by weight of elements calcium (Ca), iron (Fe), silicon (Si), carbon (C), hydrogen (H), and oxygen (O), with an empirical formula of FeₓSi_{γ}OzCₖHwCa_{I} wherein the composite material is produced by processing a versatile feedstock through an industrial digestive system.

3. A method for optimizing transformation processes in an industrial system, the method comprising: receiving input data that includes: a set of input materials to be processed; each material having an initial state DinitialD_{\text{initial}}Dinitial; and desired output objectives for the transformation processes; defining a set of candidate transformation processes available within the industrial system, each transformation process associated with variables and parameters applicable to the input materials; inputting the input data and transformation processes into a machine learning model trained using training data that includes: a plurality of input materials and their initial properties DinitialD_{\text{initial}}Dinitial, processing parameters; conditions for the transformation processes; and observed final states Dfinal_observedD_{\text{final\_observed}}Dfinal_observed of the input materials after undergoing the transformation processes; utilizing an action tensor (map) ε, referred to as the transformation tensor, which encapsulates the multi-dimensional effects of the transformation processes on various material properties from the initial state DinitialD_{\text{initial}}Dinitial to the final state DfinalD_{\text{final}}Dfinal, the tensor being a high-dimensional array where each element is associated with a specific transformation process PnP_nPn applied to a particular material mmm and affecting a certain property iii, the indices mmm, iii, and PnP_nPn representing dimensions in the material space, property space, and process space, respectively; wherein the action tensor serves to: map the effects of each transformation process across various materials and their properties; create a comprehensive model of potential outcomes; represent cumulative effects where the impact of sequential transformations on a material's property is accumulated across the process chain; and account for interactions between different processes where the effect of one transformation may depend on preceding transformations and combinations of transformations; receiving from the machine learning model predicted final states Dfinal_predictedD_{\text{final\_predicted}}Dfinal_predicted of the input materials after processing through the transformation processes utilizing the action tensor; calculating optimal input material proportions and process configurations by minimizing a composite score function that evaluates the effectiveness of the transformation processes based on: weighted criteria W={w1, w2, . . . , wm}W=\{w_1, w_2, . . . , w_m\}W={w1, w2, . . . , wm} applied to selected criteria Ck=\{ck1, ck2, . . . , ckm}C_k=\{c_{k1}, c_{k2}, . . . , c_{km}\}Ck={ck1, ck2, . . . , ckm}; and estimations of output products derived from input material proportions X=\{x1, x2, . . . , xl}X=\{x_1, x_2, . . . , x_I\}X=\{x1, x2, . . . , xl} and a matrix A=[aij]A=[a_{ij}]A=[aij] quantifying output amounts from input materials; generating an optimized configuration that includes: the optimal set of transformation processes PoptimalP_{\text{optimal}}Poptimal; the optimal proportions of input materials XoptimalX_{\text{optimal}}Xoptimal; and the optimal variables and parameters VoptimalV_{\text{optimal}}Voptimal for the processes; outputting, via control system, data representing the optimized configuration for implementation in the industrial system, thereby improving process efficiency and achieving the desired output objectives; wherein the action tensor ε integrates empirical data and machine learning predictions to optimize the process parameters, ensuring the conversion from DinitialD_{\text{initial}}Dinitial to DfinalD_{\text{final}}Dfinal is as efficient and effective as possible by simulating and predicting the outcomes of the industrial processes based on theoretical modeling and experimental data.

4. A control system for the optimization of a waste reclamation system that operates within a digital environment comprising; at least one CPU and memory that operate to store and execute computer code algorithms, or instructions; at least one sensor; at least one actuator; and an optimization module that utilizes data captured from the sensors to activate at least one the actuators; wherein the optimization module uses data from the sensors as input to an adaptive learning system that utilizes a machine learning engine and data previously captured by the sensors to identify input waste, direct the input waste through a process determined by the optimization module to operate the waste reclamation system.

5. A method of transforming an input, wherein the input comprises waste materials, molecular or elemental feedstocks, or other raw materials of varied composition in a liquid, solid, or gaseous state, into one or more usable products, comprising: inputting the input; identifying the composition or properties of the input via one or more sensors; Submitting data representing the identified composition or properties to an optimization module, which then determines at least one processing pathway for yielding one or more usable products; actuating transportation mechanisms, including doors, conveyors, pipes, valves, or hermetically sealed channels, to direct the input along the processing pathway determined by the optimization module; monitoring the progress of the input along the processing pathway, including intermediate and output characteristics, via real-time feedback from the sensors or historical data; and dynamically altering the processing pathway or operational parameters based on the monitored data to enhance efficiency, output quality, and resource recovery, thereby transforming the input into the one or more usable products.

6. The system of claim 1, wherein the input waste materials include municipal solid waste, industrial waste, agricultural biomass, hazardous materials, and radioactive materials, categorized based on their physical and chemical properties, and wherein the system incorporates safety measures and protocols for the processing of hazardous and radioactive materials.

7. The system of claim 1, wherein the control system comprises advanced machine learning algorithms specifically designed to analyze input characteristics, predict optimal transformation sequences, and dynamically adjust processes for enhanced resource recovery and conversion efficiency.

8. The system of claim 1, wherein the control system includes algorithmic differentiation for multi-input, multi-output processing, enabling the system to handle diverse waste inputs and manage multiple outputs simultaneously, tailored for varied industry applications, and capable of adjusting processing parameters to produce outputs such as biofuels, biogas, chemical substances, bioethanol, biomethane, and composite materials with thermoplastic properties.

9. The system of claim 1, wherein the control system includes a data analytics module that processes historical and real-time data, supports predictive modeling and predictive maintenance, providing insights to enhance operational longevity, reduce downtime, and optimize performance.

10. The system of claim 1, wherein the system includes cyber-physical integration with a software interface configured for remote communication, facilitating integration with IoT-enabled waste management systems, enabling remote monitoring, data-driven insights, and automated adjustments, and is compliant with Industry 4.0 standards for advanced industrial automation and data exchange.

11. The system of claim 1, wherein the system's scalable and modular architecture supports various operational scales, from pilot models to large industrial setups, featuring modular processing units such as pyrolysis and fermentation chambers, allowing for the addition or removal of processing stages based on specific needs and desired outputs.

12. The system of claim 1, wherein the system includes real-time monitoring and feedback mechanisms ensuring adaptive control of operational parameters, with sensor-based monitoring gathering data on input, process, and output characteristics for automatic adjustments to optimize processing conditions, maintain consistent production quality, and enhance system responsiveness.

13. The system of claim 1, wherein the hardware system includes automated sorting and mechanized waste processing, utilizing movable doors, hydraulic systems, mechanical force applications, pipes, and hermetic hydraulics to streamline and optimize sorting and processing, ensuring efficient and continuous flow between the plurality of stages.

14. The system of claim 1 wherein the control system alters the path of the input waste from one stage to the next or the sequence of the plurality of operations within each of the plurality of stages to optimize the output therefrom.

15. The system of claim 1 wherein the input waste is transported as a paste or semi-solid between the plurality of stages via pipes, doors, hermetic hydraulics, or the like.

16. The system of claim 4 wherein the control system comprises at least one database that is digitally accessible to the optimization module and contains criteria for the determination of outputs from the waste reclamation system.

17. The system of claim 4 wherein the optimization module comprises a feedback loop that determines an action for the waste reclamation system, causes the action to occur, measures the output, and stores data taken from the output for subsequent use to alter the determined action of the waste reclamation system.

18. The method of claim 5, wherein the optimization module employs a machine learning model trained on data derived from transformations of both waste materials and refined feedstocks, enabling the model to continuously improve predictive accuracy and processing pathways for a wide range of input types.

19. The method of claim 5, wherein the one or more usable products are selected from the group consisting of: fuel oil and biofuels, biogas, fuel gas, reconstituted chemicals or recovered elements; composite materials, incorporating but not limited to thermoplastic matrices and reinforcing fillers; and other industrially or commercially valuable substances; wherein the method is adapted to produce said products from both waste and non-waste inputs.

20. The method of claim 5, wherein the optimization module applies algorithmic differentiation and predictive modeling in response to the monitoring step, which includes integrating data from multiple sensor modalities, including but not limited to optical, spectroscopic, thermal, mass-flow, chemical composition, and microbial activity sensors to anticipate variations in input characteristics, thereby maintaining stable operation and consistent product quality across diverse input types, including previously unclassified waste and standardized feedstocks.
